(19) United States
(12) Patent Application Publication
(10) Pub. No.: US 2023/0181121 A1
(43) Pub. Date: Jun. 15, 2023

(54) SYSTEMS AND METHODS TO PREDICT AND MANAGE POST-SURGICAL RECOVERY

(71) Applicant: NANOWEAR INC., Brooklyn, NY (US)

(72) Inventors: Vijay Varadan, State College, PA (US); Prashanth Shyam Kumar, Danville, PA (US); Mouli Ramasamy, State College, PA (US); Pratyush Rai, State College, PA (US); Venkatesh Varadan, Brooklyn, NY (US)

(73) Assignee: NANOWEAR INC., Brooklyn, NY (US)

(21) Appl. No.: 17/947,740

(22) Filed: Sep. 19, 2022

Related U.S. Application Data
(60) Provisional application No. 63/245,477, filed on Sep. 17, 2021.

(51) Int. Cl. A61B 5/00; A61B 5/0205; A61B 7/00; A61B 5/265

(52) U.S. Cl. CPC A61B 5/7275; A61B 5/0205; A61B 7/00; A61B 5/726; A61B 5/7267; A61B 5/265; A61B 2562/0285; A61B 2505/05; A61B 5/318

(57) ABSTRACT

The present invention relates to systems and methods to manage and predict post-surgical recovery. More specifically, the disclosure generally relates to systems and methods for post-surgical intervention planning, support, follow-up, patient compliance, recovery prediction and tracking, and potential treatment modifications.

This application claims priority to U.S. Provisional Application Ser. No. 63/245,477, filed Sep. 17, 2021, entitled Systems and Methods To Predict and Manage Post-Surgical Recovery, the entire disclosure of which is hereby incorporated by reference.

### FIELD OF INVENTION

The present invention relates to systems and methods to manage and predict post-surgical recovery. More specifically, the disclosure generally relates to systems and methods for post-surgical intervention planning, support, follow-up, patient compliance, recovery prediction and tracking, and potential treatment modifications.

### BACKGROUND

Post-surgical recovery is an essential part of the surgical process. It ensures that there are lower risks of long-term physical complications, as well as short-term complications that can worsen a patient's condition. Optimal recovery increases patient satisfaction, outcomes, and reduces the cost of care. Tracking physiological data is essential in this process as it allows physicians to be aware of any complications that may arise during this stage.

There are a wide range of vitals that physicians track post-surgically to ensure proper recovery for the patients. The most important vitals that are generally tracked are ECG, bioimpedance, heart sounds, blood oxygenation, pulse rate, activity levels and the like. Additional vitals such as capillary refill time, volume of pulse, rhythm of pulse, skin turgor, diaphoresis, and others may also be tracked.

Post-surgical tracking can be done in a variety of different methods, each with different uses, levels of specificity and accuracy, and advantages and disadvantages. There are approaches that provide software-based solutions to post-surgical recovery tracking, involving applications that use surveys, communication methods, and informational material for patients. Examples include a mobile based tracking application that can be used for a variety of different surgeries. This focuses on patient engagement by gathering evidence regarding their pain and compliance through validated surveys. They also include daily health checks, educational information for patients, and the ability to send the physician pictures of things such as incisions and stomas for proper recovery. Through this, they have been able to reduce patients' length of stay, readmission rates, hospital visits, phone calls, and save money in a variety of different hospitals that use their technology. Another example utilizes Artificial Intelligence (AI) and allows physicians to remotely track their patients after surgery. Patients receive personalized daily care reminders, educational resources, and an AI-driven check-in tool that captures their physical and emotional symptoms. This application collects information on adherence, SDOH (Social Determinants of Health) issues, ADL (Activities of Daily Living) support needs, well-being trends, spiritual health needs and more. Its applicability goes beyond post-operative surgical care, and can apply to home health, hospice, palliative care, oncology, surgical care, and behavioral health. Specific to surgical care, it can reduce cancellations and reschedules with pre-surgery symptom monitoring and surgery pre-clearance, assess post-surgical mobility risk and barriers to smooth recovery, and deliver personalized care instructions including wound care and physical therapy. It can also reduce readmissions with automated monitoring for signs of post-op infection and fall risk remotely monitor incision site healing progression and identify issues proactively, bolster and track post-op mobility progress, and facilitate measurement of patient reported outcomes and satisfaction. Another example is a web-based platform that monitors patient progress from the operating room to the PACU (Post-Anesthesia Care Unit). It integrates available data, automatically determining when a patient enters the PACU. An automated paging system alerts clinical unit managers to ‘pull’ their patients from the PACU after a set recovery period. The PACU length of stay was reduced using this solution. This is different in comparison to other solutions as it only focuses on its applications within the hospital rather than home/remote care. Another example include sending pictures of incisions to the surgical team, being able to contact and communicate with the surgical team, logging total drain output, attaining information about their condition and the surgery they received, and tracking progress over time.

Compliance to prescribed behavioral patterns following a surgical procedure is an important component of the recovery process. Recovery can be delayed or lead to complications due to non-compliance by the patient. Depending on the type of procedure, the prescribed behavioral pattern may include minimum durations of light activity, such as walking or movement of the affected surgical area, and avoidance of certain postures. Altitude relative to sea level and geographic location impacts blood oxygenation and sympathetic responses such as vasodilation to compensate for any reduction of oxygen saturation in the blood due to lowering the concentration of oxygen at higher altitudes, or presence of endemic pollutants, gases, or particles that may affect the rate or trajectory of recovery of the patient. Similarly, there are hardware-based approaches as well. These typically fall into two categories, the first being wearable devices and/or at-home or hospital-based patient monitors, and the second being implantable devices. Devices such as these can be used to track heart rate, steps and other health indicators, and ECG to monitor patients after their surgery. These reports are often sent to the patient's physician for review and used for optimal recovery. Physicians also use activity trackers to numerically track activity after surgery. This can show exactly how much function a patient has regained and if it occurs during the recovery period. Other approaches could include tracking skin temperature, auscultation, heart rate, oxygen saturation, heart rate variability, hemoglobin/hematocrit, volumetric flow, blood pressure, potassium, range of motion (ROM), ambulation, exercise compliance, and wound site temperature trends.

There are numerous advantages and disadvantages of each type of method. Software solutions could be a cost-effective method of tracking and can therefore be more accessible to many people. However, their efficacy is low, and these solutions cannot produce accurate quantitative data based on the actual physiology that would be essential for physicians during the patients' recovery period. Implantable devices, on the other hand, can be accurate but are often highly expensive, cannot be updated, upgraded, or repaired easily, and require an invasive procedure to be implanted in the body. Lastly, wearables and other patient monitors offer a good middle ground as they have the ability to be cost effective while also allowing for accurate quantitative results. Overall, post-surgical recovery tracking is very important and is currently a large and growing market. While there are multiple approaches, they have myriad of disadvantages as outlined earlier including but not limited to lack of accuracy, track few vitals, do not track vitals at all, high cost, poor ease of use, etc.

### SUMMARY OF THE INVENTION

Although the aforementioned approaches can give some insights on how the patient feels and allow doctors to view their patient's progress visually, they provide little to no information of the actual physiological condition of the patient. The goals of post-surgical tracking are therefore not achieved properly as patients will still have to stay in the hospital to track their biometrics and vitals.

Therefore, there is still a need for systems and methods for surgical and interventional planning, support, postoperative follow-up, and functional recovery tracking. The present invention relates to systems and methods to manage and predict post-surgical recovery. More specifically, the disclosure generally relates to systems and methods for post-surgical intervention planning, support, follow-up, patient compliance, recovery prediction and tracking, and potential treatment modifications. A user, relating to a patient, physician, medical professional or the like can receive predictive outcomes about a patient, based on the specific surgery or treatment or a plurality of multiple surgical procedures or measured physiological and biological data, and historic patient medical data and records. Multiparametric wearable device data that simultaneously captures heart sounds, ECG, thoracic impedance, activity, and posture has not been used to interpret and track the perioperative status of patients around the time (a matter of few days to 6-12 months before, during, and after) of a surgical procedure.

It is an object of the present invention to provide an assessment of post-surgical intervention planning, support, follow-up, patient compliance, recovery prediction and tracking, and potential treatment modifications.

It is a further object of the present invention to provide a system capable of collecting data to manage and predict post-surgical recovery. It is a further object to more specifically provide systems and methods for post-surgical intervention planning, support, follow-up, patient compliance, recovery prediction and tracking, and potential treatment modifications where a user, relating to a patient, physician, medical professional or the like receives predictive outcomes about a patient, based on the specific surgery or treatment or a plurality of multiple surgical procedures or measured physiological and biological data, and historic patient medical data and records.

It is additionally an object of the present invention to provide a method of collecting and assessing data relating to post surgery intervention to be used for personalized patient care. It is a further object of the present invention for the method to include treatment of post-surgery complications based on the assessment of the data.

In accordance with an embodiment of the present invention, an integrated system for post-surgical intervention planning, support, follow-up, patient compliance, recovery prediction and tracking, and potential treatment modifications is provided. In accordance with another embodiment of the present invention, an integrated system for assessing and treating complications of surgery is provided.

In accordance with a further embodiment of the present invention, the system may detect, process and report various physiologic and chemical parameters such as biomarkers measured at various areas of the body such as lactate, pH, alcohol, nicotine, sodium, glucose, urea, chloride, discrete blood, interstitial fluid, cerebrospinal fluid (CSF) biomarker panels, and metabolic panels, urinalysis panels, data or derivatives from geographic location and altitude metrics, data or derivatives from patient historic data and combinations thereof. In accordance with a further embodiment of the present invention, the system may make treatment determinations based on these findings.

In accordance with an embodiment of the present invention, the system may detect one or more physiological values from a patient such as physiologic data including pH, temperature, moisture level, microbial activity, brachial blood pressure at the right or left arm, aortic blood pressure, left ventricular end diastolic pressures, pulmonary artery and venous pressures, measurements of chest circumference around the bottom of the sternum, maximum oxygen consumption VO2 max, and hemodynamic metrics such as cardiac output and stroke volume, and providing post-surgical intervention planning, support, follow-up, patient compliance, recovery prediction and tracking, and potential treatment modifications. In accordance with another embodiment of the present invention, the system may compare one or more detected physiological values to predetermined physiological values in order to obtain a comparison result in real time.

In accordance with certain preferred embodiments of the present invention, the physiological values may be detected by one or more sensors and electronics. In accordance with certain further embodiments of the present invention, the sensor(s) may be nanostructured working electrode (WE) and counter electrode (CE), and silver-silver chloride reference electrode (RE). In accordance with certain embodiments of present invention, the sensor(s) may be an array connected to an electronics module that acquires sensor signals, sends electrical stimuli and communicates wirelessly to mobile device at programmable time intervals.

In accordance with certain further embodiments of present invention, the system may compute a composite score for wound healing and to determine the frequency and magnitude of delivery of therapeutics. In a preferred embodiment of the present invention, the assessment is presented as a numeric, symbolic, image, or video.

In accordance with another embodiment of the invention, the system may also include components to treat other conditions such as high or low blood pressure, bleeding or hemorrhage, bacterial infections, fever, inflammations, sores, and pain, and sensing components for sensing one or more values of one or more physiological or chemical parameters of a patient, such that the sensing, analyzing and treatment of abnormal physiological and chemical parameters is integrated.

In an embodiment of the present invention, a method for assessment of a patient during perioperative care is provided which comprises the steps of:
a) collecting a plurality of data such as skin temperature, auscultation, heart rate, oxygen saturation, heart rate variability, hemoglobin/hematocrit, volumetric flow, blood pressure, potassium, range of motion (ROM), ambulation, exercise compliance, and wound site temperature trends and combinations thereof;
b) conditioning the input data using an engineering system through e.g., filtering methods to remove undesired noise or patterns that are irrelevant to the estimation of patient status assessment within the input data obtained, with appropriate filtering methods including finite impulse response filters, infinite impulse response filters, recursive filters, wavelet denoising, signal smoothening using running averages or autoregressive moving averages, and adaptive filters, ideal filters and optimal filters or combinations thereof;
c) extracting a plurality of features from the plurality of data;
d) transforming the features into qualitative or quantitative metrics using an engineering method such as transformation of the input data into a subset or an equivalent set of data that does not lose any information content from the original data, e.g. principal components determined through a process of principal component analysis which yields a transformation matrix that can transform input data into the principal components basis which are multidimensional with each principal component representing a dimension or transformations can also be reductive, and components that do not contribute to the information or variability of the overall set of features can be discarded so that fewer components are retained leading to reduction in the dimensionality;
e) assessing the patient using the metrics of step d, wherein e.g., a single quantifiable metric may describe, as a result of step e, a representation of the time varying status of a patient as an indication of whether there has been a change in the overall status of the patient as a cumulative effect of changes that are manifesting among the metrics that were computed and chosen as relevant to tracking recovery after a surgery.

In certain embodiments of the present invention, the method uses Method 14 in FIG. 5 to determine a single metric that is a patient status assessment with the input data transformed into a set of features that are relevant to patient status assessment. Such a list of features is presented in Table 1 as an exemplary embodiment wherein each feature is obtained for a specific time of measurement over an epoch such as using input data obtained over a minute. The features are then subjected to clustering methods which cluster similar instances of features across epochs. The clustering methods then assign cluster numbers to each cluster. Once each feature is assigned to a cluster, the total number of feature instances belonging to each cluster is counted and referred to as cluster membership. This calculation is performed as part of translation of transformed data into a metric. The quantified change metric which is the same as patient status assessment is the ratio of the difference in cluster membership to the total number of observations for each cluster. Thus, a patient status assessment as a single number or metric is obtained.

In a further embodiment, the method provides further actions such as planning, support, follow-up, patient compliance, recovery prediction and tracking, potential treatment modifications and combinations thereof. In additional embodiments, the input data includes historic patient data and patient questionnaires.

In an embodiment of the invention, the plurality of input data collected can be data or derivatives (Derivatives here mean the same as features extracted where specific characteristics of the input data is calculated algorithmically from the unmodified raw input data. For example, electrical activity-based measurements such as ECG (electrocardiogram), EMG (electromyogram), and EEG (electroencephalogram) have patterns that are characteristic and relate to certain underlying physiological phenomena observed in patients that are relevant to post-surgical recovery process. More concretely, heart health can be assessed by extracting various waveform characteristics from the ECG such as QT waveform intervals, heart rate variability based on the change in the time interval between successive R peak waveforms, or in the case of EMG, the amplitude and frequency versus power distributions of the EMG waveforms reveal muscle strength and recovery patterns in muscle groups where the EMG is measured. EEG is used extensively in sleep studies to gauge the quality of sleep and any patterns of abnormal breathing. Sleep is a very important predictor of surgical recovery trajectory whether better or worsening patient status. Data or derivatives can be obtained from a method such as electrical activity based metrics which are electrical potential of currents measured within the body that reflect physiological function of different organs or muscles, such as the ECG for the heart muscle as a whole, EMG for skeletal muscles, and EEG for the holistic functioning of the neurons in the brain, bioimpedance based metrics which are electrical measurements of the tissue composition at or near the site where surgery was performed for specific monitoring of healing. This can also extend to monitoring overall body composition monitoring to ensure that the patient is compliant to recovery guidance from physicians to maintain a certain level of body weight and fat percentage. The bioimpedance based metrics are specifically measured by applying a small current in the milliamp range at a range of frequencies following a periodic waveform such as a sinusoidal wave or square wave between two electrodes or nanosensors placed on the body and using another pair of nanosensors or electrodes to measure the electric potential caused by the flowing current between the previous pair used to apply the currents. Such measurements can be obtained using instrumentation such as transimpedance calculators and phase-locked-loops to tune and measure currents at certain frequencies. The Nanowear® SimpleSense™ device performs such measurements, with mechanical action metrics such as mechanical-, impedance-, electrical-based metrics obtained simultaneously using a single measurement instrument or at the same time from separate measurement instruments being preferred. Also applicable are goniometric measurements of all joint angles, absolute inclination, and orientation of different parts of the body in multiple axes, photoplethysmographic measures at various sites on the body such as fingers, toes, ankle, foot, arms, thorax, neck, and forehead, sounds such as heart sounds, lung sounds, gastrointestinal sounds, and joint sounds, blood oxygen levels, skin or body temperatures measured at different locations of the body such as extremities, thorax, abdomen, and head, parameters such as lactate, pH, alcohol, sodium, glucose, urea, chloride, discrete blood, interstitial fluid, cerebrospinal fluid (CSF) biomarker panels, metabolic panels, liver function panels, kidney function panels, and urinalysis panels, and combinations thereof, geographic location and altitude metrics, patient historic data, patient questionnaires, metrics such as risk stratification by means of a hazard ratio or index, or a recovery percentage score indicative of change and trajectory of change in a patient's status around an index event which involves a surgical intervention or combinations thereof. An index event is different from the hazard ratio or index. An index event is a point in time when medical activity has occurred. In this case, reference is specifically made to a surgical intervention as an index event to indicate a point in time before and after which constitutes the time frame or surgical perioperative care. In preferred methods, goniometric measurements specifically are important.

In another embodiment of the present invention, a method is provided for assessment of a perioperative patient, comprising:
a plurality of improvements, conditioning, and correction systems and methods to account for data quality and confounders;
a plurality of feature extraction systems and methods to extract a plurality of features or data and model assessment from a plurality of measurement devices, historic patient data, and patient questionnaires;
a plurality of systems and methods for the signal and model assessment to provide inputs for model improvements, conditioning, and correction; and
obtaining an assessment of a perioperative patient, which may also include model improvements, conditioning and correction of care.

In further embodiments, the method includes a method of personalization of the assessment. The output of the patient status assessment is improved using data collected from the patient for whom the assessment is being performed with the personalization methods improving the accuracy of the patient assessment, whether it is a recovery score or a risk score. These methods are applicable specifically to the case where improvements in the accuracy of the assessment is sought for a specific individual referred to as the process of personalizing the models. A patient's input data before a surgical procedure can e.g., be used as baseline and each data available as input from that time onward, through the procedure, and afterward is compared to the baseline to track recovery as a relative change from pre-surgical baseline.

The baseline is only relevant to a specific patient, so it is personalized to that patient. An example of a method of personalization comprises:

a. performing one or more improvement, conditioning, and/or correction methods or processes to account for data quality and confounders;
b. performing data conditioning methods and processes for data conditioning and preparation of the data;
c. performing one or more feature extraction methods and processes to extract a plurality of features for signal and model assessment from one or more measurement devices and historic patient data;
d. performing one or more feature selection methods and processes for selecting features that are relevant to assessment;
e. performing normalization, combination and/or transformation methods and processes for the signal and model assessment to provide inputs for the assessment prediction model for improvements, conditioning, and correction; and
f. providing a post-surgical assessment which can include intervention planning, support, follow-up, patient compliance, recovery prediction and tracking, and potential treatment modifications.

In another embodiment, the method further includes a continuous improvement of the assessment accomplished through calibration. Concretely, a subset of the input data such as patient recovery questionnaires can be completed by the patient at regular intervals and this questionnaire data or answers can serve as input data to calibrate the assessment produced by the method followed in an embodiment. For example, if the patient questionnaire entries reveal that the patient is not recovering as quickly as estimated by the method for estimating recovery status, then the recovery status can be altered by weighting the values with a penalty so that the recovery is not overestimated.

An example of a method of continuous improvement method comprises:

a. performing improvements, conditioning and/or correction methods and processes to account for data quality and confounders;
b. performing feature extraction methods and processes to extract a plurality of features for signal and model assessment from a plurality of measurement devices and historic patient data;
c. performing feature selection methods and processes for selecting features that are relevant to the assessment;
d. performing one or more normalization, combination, and/or transformation methods or processes for the signal and model assessment to provide inputs for the assessment prediction model for improvements, conditioning, and correction; and
e. providing a post-surgical assessment which can include intervention planning, support, follow-up, patient compliance, recovery prediction and tracking, and potential treatment modifications.

In an embodiment of the present invention, a pre-trained model (i.e., a pre-trained neural network) on a population is further trained with additional input data from an individual to generate a surgical recovery assessment model that is unique for that individual. The output of the patient status assessment is improved using data collected from the patient for whom the assessment is being performed. All personalization methods are designed to improve the accuracy of the patient assessment whether it is a recovery score or a risk score. In preferred embodiments, the population is a large population comprising at least 50 patients each for a chosen surgical procedure such as cataract removal, C-section, joint replacement, bone repair, stent procedure, Coronary Artery Bypass Graft (CABG), percutaneous coronary intervention, angioplasty and atherectomy, and aesthetic surgery such as facelift, breast augmentation or reduction, liposuction, and tummy tucks. In certain preferred embodiments, the population comprises at least 100 patients and up to 10000 patients. The method can be repeated and continuously improved upon by adding further input data obtained from the patient each time the method is repeated to generate a prediction model that is unique for the patient. In certain preferred embodiments, the method is repeated and each time the method is repeated, updated input data obtained from the patient is added to step (a) resulting in an assessment model that is unique for the patient. In certain preferred methods, the further input data such as patient reported outcomes, physiological measures, and combinations thereof.

In certain embodiments of the present invention, the method predicts a degree of certainty from about 75% to about 95% for each of the predicted outcomes associated therewith, each of the degrees of confidence based at least on the predicted data or the historical data regarding signal and model assessment methods and processes on a plurality of patients. Predicted outcomes may be binary reflecting whether the patient recovered to a known baseline state prior to a surgical procedure. Concretely, “1” may be recovered and “0” maybe not recovered. In certain preferred embodiments, the predicted outcome can be a continuous number from 0 to 1 or 100 with several of the extracted features accounting for this score such as a weighted sum of attributes, e.g., wound healing status, presence of infection, pain level, and patient reported recovery questionnaire scores. The method of obtaining the confidence value can be from historical data belonging to a particular population of patients undergoing a specific procedure such as males between 60 and 65 years old undergoing a CABG procedure and the amount of variation that was observed in their recovery times. The confidence in a predicted outcome, a score, or a time till recovery will be the mean of the distribution of potential values (e.g. from one of the methods used in FIG. 5 or 7) and the variance will reflect the variance seen in the historical data.

In embodiments of the present invention, generative neural network that consists of structures involving a generator component and a discriminator component, such as a Generative Adversarial Network (GAN) is incorporated into the method. The generator component is a neural network that takes as input a random input signal that can be generated using a pseudo random generator with a white noise distribution and generates the input data that is part of the training data set. This component generates the input data and is trained to generate the input data with minimal error. The discriminator component is also a neural network that takes as input the output of a generator component and determines whether the output is acceptably close to the input data using a cost function that evaluates the amount of error. Given a training set, this technique learns to generate new data with the same statistics as the training set. It is possible, for instance, to use a GAN and its discriminator output to extract features. For example, a GAN trained on photographs can generate new photographs that look at least superficially authentic to human observers, having many realistic characteristics. The core idea of a GAN is based on the “indirect” training through the discriminator, another neural network that can tell how “realistic” the input seems, which itself is also being updated dynamically. This means that the generator is not trained to minimize the distance to a specific image, but rather to fool the discriminator, hence adversarial. The input data applied to the discriminator generates a set of features that can be used to train another neural network or machine learning model to predict post-surgical recovery or risk of complication.

In another embodiment of the present invention, a method is provided for improving a patient's recovery using assessment predictions generated during perioperative care, wherein the assessment predictions are further configured as inputs to develop time series forecasting model such as Autoregressive models (AR), Moving Average (MA), Autoregressive Moving Average (ARMA), Autoregressive Integrated Moving Average (ARIMA), and Seasonal Autoregressive Integrated Moving-Average (SARIMA), using the respective model architectures and parameter optimization procedures that are known to those skilled in the art to predict an assessment for a set of possible further surgeries for the patient at a specific point in time after the initial surgery for which the predictions were made. The outputs of the forecasting models are potential future values of assessments that could be obtained using historically known assessments as input; wherein the further configured assessment predictions for possible further surgeries are configured to output both a mean prediction and a standard error around the mean value so that the standard error may be used to predict a degree of confidence such as a value for a recovery score and a confidence of 95% indicating that there is 95% confidence that the predicted recovery score or assessment will be within the predicted range for a future surgery. In preferred embodiments, the degree of confidence is from about 75% to about 95%.

In a further embodiment of the invention, the assessment predictions are based at least on measured data from a patient, derived data, extracted data, patient historic data, and/or patient questionnaire data. In a further embodiment, a report is generated assessing the status of the patient. In a further embodiment, the assessment prediction also provides treatment recommendations based on input data obtained from the patient post-surgery combined with the assessment predictions. In another embodiment, the assessment prediction also provides intervention planning, support, follow-up, patient compliance, recovery prediction and tracking, and potential treatment modification options.

In certain embodiments of the present invention, a nanosensor is used to obtain certain of the features for example, a thermosensitive nanosensor. For example, a thermosensitive nanosensor can comprise a substrate sandwiched between the insulating layer and a conductive layer; vertically standing nanostructures attached to the substrate; a conductive material on top of the nanofiber surface; a thermosensitive hydrogel layer on top of the conductive layer; and a cover layer on top of the thermosensitive hydrogel to prevent loss of moisture and mechanical stress. Alternatively, the thermosensitive nanosensor can include a substrate having a plurality of vertically standing nanostructures attached thereto, the plurality of vertically standing nanostructure being covered with a conductive material to form conductive coated nanostructures; a thermosensitive hydrogel adjacent to the plurality of conductive coated nanostructures; and a cover layer on top of the thermosensitive hydrogel to prevent loss of moisture and mechanical stress. The substrate may include a fabric sandwiched between an insulating layer and conductive layer. Disclosure of such thermosensitive nanosensors is found e.g. in U.S. Publication No. 20210000417, incorporated by reference herein.

In certain embodiments, the features are obtained from wearable remote electrophysiological monitoring system such as that disclosed in U.S. Publication No. 20160183835, incorporated by reference herein. Such a system may include a garment having at least one nanostructured, textile-integrated electrode attached thereto; a control module in electrical communication with the at least one nanostructured, textile-integrated sensor, and a remote computing system in communication with the control module. In other embodiments, the features are obtained using a non-invasive, wearable and portable medical device for evaluation and monitoring of a patient, such as the wearable textile-based harness including an adjustable elastic horizontal band and an adjustable elastic vertical band as disclosed in U.S. Patent Publication 20210177335, the disclosures of which are incorporated by reference herein. In other embodiments, the features can be obtained through a bandage system capable of collecting data such as the system disclosed in U.S. Patent Application No. 63/174,721, the disclosures of which are incorporated herein. The system may detect one or more physiological values from the wound of the patient. The system may compare one or more detected physiological values to predetermined physiological values in order to obtain a comparison result in real time. The physiological values may be detected by one or more sensors and electronics. The sensors may be nanostructured working electrode (WE) and counter electrode (CE), and silver-silver chloride reference electrode (RE). The sensor(s) may be an array connected to the electronics module that acquires sensor signals, sends electrical stimuli and communicates wirelessly to mobile device at programmable time intervals. The system may compute a composite score for wound healing and to determine the frequency and magnitude of delivery of therapeutics. Suitable sensors for use in the present invention are described in U.S. patent application Ser. No. 16/916,843, the entire disclosures of which is hereby incorporated by reference. The teaching of U.S. patent application Ser. No. 17/941,880 is also incorporated by reference.

In certain embodiments of the invention, physical and chemical features of interest are also obtained by the above methods to e.g. monitor the heart activity, monitor or assist wound healing or other useful purpose. For example, the system and method of the present invention could be used in association with a wearable remote electrophysiological monitoring system which includes a fully wearable textile integrated real-time ECG acquisition system with wireless transmission of data for the continuous monitoring of e.g. athletes during training and competition. In certain further embodiments, the system of the present invention can make treatment determinations based on the assessment findings, or when applicable, the other physiologic or chemical findings.

In an embodiment of the present invention, a method is provided for assessment of a patient during perioperative care comprising the steps of:
a) selectively obtaining a plurality of input data from one or more measurement devices, selection and collection methods and/or processes;
b) subjecting the input data to a transformation selected from the group consisting of conditioning, feature engineering and combinations thereof;
c) translating the transformed input data into metrics; and
d) using the metrics obtained in step c to obtain an assessment of the patient.

In certain embodiments, the input data is selected from past diagnoses, test results for blood biomarkers, proteins, metabolites, and/or cholesterol, biomedical vital signs collected from non-invasive medical devices, patient reported responses to Quality of Recovery questionnaires, physiological and biological data, a height of the patient, a weight of the patient, a gender of the patient, an age of the patient, a medical history and/or physical examination records of the patient, a medical status of the patient, a body mass index (BMI) of the patient, an ethnicity of the patient, a medical prescription history of the patient, a medical prescription status of the patient, types of treatments and medications received by the patient, types of medical treatments for health issues and insurance or claims information previously received by the patient, diet information for the patient, psychological history of the patient, a genetic indicator of the patient, biomarkers of the patient, the Electronic Medical Record (EMR) of the patient information and combinations thereof. In other embodiments, the physiological and biological data is selected from Electrocardiogram (ECG), Electromyogram (EMG), Electrooculogram (EOG), Electroencephalogram (EEG), Galvanic Skin Resistance (GSR), Goniometric measurements of all joint angles, absolute inclination, and orientation of different parts of the body in multiple axes, photoplethysmographic measures at various sites on the body, heart sounds, lung sounds, gastrointestinal sounds, joint sounds, acoustic impedance, electromagnetic impedance, ultrasonic impedance, blood oxygen levels, temperatures measured at different locations of the body, sweat biomarkers measured at various areas of the body such as lactate, pH, alcohol, nicotine, sodium, glucose, urea, chloride, discrete blood, interstitial fluid, cerebrospinal fluid (CSF) biomarker panels and combinations thereof. In additional embodiments, the input data comprises measurements made from patients reflective of physiological conditions such as Electrocardiogram (ECG), Electromyogram (EMG), Electroencephalogram (EEG), Phonocardiogram (PCG), activity and posture, sweat, blood and urine analysis results, and historical information on diagnosed conditions, past surgical interventions, and history of medications.

In embodiments of the present invention, the biomedical vital signs collected from non-invasive medical devices are e.g., selected from ECG, photoplethysmography, heart sound, activity, and calculate heart rate, respiration rate and combinations thereof. In embodiments of the present invention, the data conditioning is obtained by methods such as filtering, trend removal when there are gradual drifts in the measurement values due to the instrumentation used to perform the measurement, signal processing methods that increase the proportion of physiologically relevant data to the noise, transformations of the input data from the time domain to other domains; application of filtering techniques to segment and extract quantitative or qualitative measures correlated with physiological factors in turn correlated to patient status assessments, and neural networks. In certain embodiments, the input data is conditioned and after conditioning, the input data is prepared by translating the input data into a format that is compatible with Step 3. In certain embodiments, the input data transformation is obtained by methods e.g., selected from Fourier, wavelet, short-time Fourier, cepstral analysis, empirical mode decomposition, or wavelet decomposition. In certain embodiments, the feature engineering is comprised of feature extraction to result in features and wherein the feature extraction involves a technique or method e.g., selected from discrete Fourier and short-term Fourier transforms, discrete cosine transform, Autoregressive models (AR), Autoregressive moving average models (ARMA), classes of linear predictive coding models, cepstral analysis derived Mel-Frequency Cepstral Coefficients (MFCC), Kernel-based modeling, multiresolution analysis using discrete and continuous wavelet transformations, wavelet packet transformations and decompositions, empirical mode decompositions, power spectrum estimation using techniques that measure spectral coupling across different signal modalities, non-negative matrix factorization (NMF), ambiguity kernel functions, a subset of the layers from a pre-trained multilayer neural networks used as a transformation from input data into feature vectors in the feature space, unsupervised or supervised clustering methods like adaptive resonance-based neural networks, self-organizing maps, k-means clustering, k-nearest neighbors, Gaussian mixture models, and Naïve Bayes classifiers which group together similar feature sets (plurality of features extracted or plurality of statistically summarized inputs and assign group labels to each instance of a set of features. In certain embodiments, the feature extraction involves the use of multiresolution analysis and signal decomposition using wavelet transforms to condition the heart sound data.

In embodiments of the present invention, the assessment provides an overall metric that is reflective of the patient's state of recovery, a risk stratification score or number that is reflective of a probability or likelihood of a patient developing symptoms of a complication or risk of developing a condition that requires emergency treatment following a surgical procedure or combinations thereof. In certain embodiments, the transformation in Step b is obtained by clustering methods and the translation of the transformed input data into metrics of Step c involves a mathematical model to transform a cluster membership into a one-dimensional metric. In certain embodiments, the feature engineering comprises the steps of feature transformation and/or decomposition. In certain further embodiments, the feature engineering further comprises feature selection. In certain embodiments, the transformation and/or decomposition involves e.g., techniques selected from box cox transformation, eigen value, vector decomposition, principal component analysis (PCA), kernel PCA, truncated singular value decomposition, multidimensional scaling, isometric mapping, t-distributed stochastic neighbor embedding, wavelet denoising, neural networks and combinations thereof. In embodiments of the present invention, the method of selection e.g, is selected from measurement of mutual information using Kullback-Leibler convergence, minimum redundancy maximum relevance, impurity-based feature importance using random forest regression models, F-statistic or f-test, neighborhood component analysis, backward elimination, forward selection, permutation feature importance, factor analysis, and relief algorithm for regression.

In certain embodiments of the present invention, during step b) the input data is conditioned using an engineering system e.g., selected from filtering in time, frequency, wavelet, or other domains defined by a span of output of a convolutional neural network prior to a final layer which is a connected layer, so that the transformation does not remove any information from the data that is being transformed, then transforming the conditioned input data into qualitative or quantitative metrics using a method e.g., selected from dimensionality reduction techniques consisting of box cox transformation, eigenvalue, and vector decomposition, principal component analysis (PCA), backward elimination, forward selection, random forests impurity-based importance, permutation feature importance, factor analysis, linear discriminant analysis, truncated singular value decomposition, kernel PCA, t-distributed stochastic neighbor embedding, multidimensional scaling, isometric mapping and combinations thereof and wherein the assessment of the patient using the metrics of step d is a representation of the time varying status of a patient and indicates whether there has been a change in the overall status of the patient as a cumulative effect of changes that are manifesting among the metrics that were computed and chosen as relevant to tracking recovery after a surgery. In embodiments of the present invention, based on the assessment, the method provides further actions e.g., selected from planning, support, follow-up, patient compliance, recovery prediction and tracking, potential treatment modifications and combinations thereof. In certain further embodiments, the assessment is presented as a numeric, symbolic, image, or video.

In an embodiment of the present invention, a method is provided for assessment of a perioperative patient, comprising:
a) obtaining input data and/or derivatives;
b) performing improvements, conditioning, and/or correction methods and processes to the input data to account for data quality and confounders;
c) performing feature extraction methods and processes to the product of step b to extract a plurality of features for signal and model assessment from a plurality of measurement devices and historic patient data;
d) performing a plurality of feature selection methods and processes for selecting features that are relevant to the assessment;
e) performing one or more normalization, combination and/or transformation methods or processes for the signal and model assessment to provide inputs for the patient status assessment model for improvements, conditioning, and correction; and
f) using the output of step e to obtain an assessment of a perioperative patient.

In certain embodiments, the method further includes personalizing the assessment. In certain embodiments, the method further includes continuous improvement of the assessment through incorporation of patient specific data as part of the input data to improve the assessment. In embodiments of the present invention, a model is pre-trained on a population of at least 50 patients wherein the method is repeated and continuously improved upon by adding further input data obtained from the patient each time the method is repeated to generate a prediction model that is unique for the patient. In certain preferred embodiments, the method is repeated and wherein each time the method is repeated, updated input data obtained from the patient is added to step (a) resulting in an assessment model that is unique for the patient.

In embodiments of the present invention, the further input data e.g., is selected from patient reported outcomes, physiological measures, and psychological measures and combinations thereof. In certain embodiments, the method predicts a degree of certainty of from about 75% to about 95% for each patient status assessment associated therewith, wherein each of the degrees of confidence is based at least on the predicted data, the historical data, or patient questionnaire data regarding data and model assessment methods and processes on a plurality of patients. In certain embodiments, a generative neural network is added, wherein the generative neural network comprises a generator component and a discriminator component. In certain further embodiments, the data from step a applied to the discriminator component generates a set of outputs that are used as features that can be used to train another neural network or machine learning model.

In an embodiment of the present invention, a method for improving a patient's recovery using assessment predictions generated during perioperative care is provided, wherein the assessment predictions are further configured to predict an outcome of a set of possible further surgeries for the patient at a specific point in time after the surgery; wherein the further configured assessment predictions for possible further surgeries are configured to predict a degree of confidence for each of the assessment predictions, where the degree of confidence indicates the likelihood that the patient will achieve the assessment prediction. In embodiments of the invention, the assessment predictions are based at least on measured data, derived data, extracted data, patient historic data, and/or patient questionnaire data. In certain preferred embodiments, a report is generated assessing the status of the patient.

In certain embodiments of the present invention, the assessment prediction also provides treatment recommendations based on input data obtained from the patient post-surgery combined with the assessment predictions. In certain embodiments, the assessment prediction also provides intervention planning, support, follow-up, patient compliance, recovery prediction and tracking, and potential treatment modification options. In embodiments of the invention, input data and/or derivatives are obtained from a method e.g., selected from electrical activity based metrics, bioimpedance based metrics, mechanical action metrics e.g., selected from goniometric measurements of all joint angles, absolute inclination, and orientation of different parts of the body in multiple axes, photoplethysmographic measures at various sites on the body, heart sounds, lung sounds, gastrointestinal sounds, and joint sounds, blood oxygen levels, skin and/or body temperatures measured at different locations of the body, parameters e.g., selected from lactate, pH, alcohol, sodium, glucose, urea, chloride, discrete blood, interstitial fluid, cerebrospinal fluid (CSF) biomarker panels, metabolic panels and combinations thereof, geographic location and altitude metrics, patient historic data, patient questionnaires, metrics e.g., selected from risk stratification by means of a hazard ratio or index, or a recovery percentage score indicative of change and trajectory of change in a patient's status around an index or event which involves a surgical intervention or combinations thereof.

### BRIEF DESCRIPTION OF THE DRAWINGS

FIG. 1a shows an exemplary method to use unsupervised clusters to quantify change from one day or chosen epoch to the next measurement from an arbitrary future epoch in time.

FIG. 1b shows an exemplary method to develop a cluster classification based on a given patient's data.

FIG. 2 shows an exemplary method for qualitative change assessment of patient status from one epoch to the next, with the figure representing the density of cluster memberships on a two-dimensional self-organizing map grid, where the chosen epoch is one day.

FIG. 3a shows an exemplary method of converting a qualitative representation of a distribution of data into a numeric quantifiable metric.

FIG. 3b shows an exemplary conversion of a distribution of an observation or input data set into a quantitative numeric such as width or height of a triangle enclosing the histogram.

FIG. 4 shows an exemplary image showing an embodiment of the risk or hazard score presented in a clinician dashboard for a patient being monitored remotely, with the dashboard also displaying several other vital signs that are important for remote monitoring, e.g., heart rate, blood pressure, and respiration.

FIG. 5 is a chart showing a plurality of methods to formulate model architecture that predict patient status, or trajectory towards recovery after surgical intervention using unsupervised machine learning methods.

FIG. 6 shows an exemplary image showing clustering of aggregated input data in 84 dimensions as viewed on a self-organizing map grid of 30 by 30 neurons.

FIG. 7 is a chart showing a plurality of methods to formulate model architecture that predict patient status, or trajectory towards recovery after surgical intervention using supervised machine learning methods or combinations of supervised and unsupervised machine learning methods.

### DETAILED DESCRIPTION OF THE EMBODIMENTS

Generally, following standard practice in the care for patients, an assessment of a patient may include a complete medical history, medical tests, a physical exam, a test of learning skills, tests to find out if the patient is able to carry out the tasks of daily living, a mental health evaluation, and a review of social support and community resources available to the patient. In the context of surgical recovery, assessments can include a variety of activities such as collecting patient reported feedback regarding pain levels, patient's subjective description of whether they have recovered after the surgery to a state that was evident before the surgery, obtaining an overall metric that is reflective of their state of recovery such as a patient recovery score that reflects a chance or probability of recovery of a patient to a pre-surgical state following a surgery, or a risk stratification score or number that is reflective of a probability or likelihood of a patient developing symptoms of a complication or risk of developing a condition that requires emergency treatment following a surgical procedure. In the present invention, the assessment provides an overall metric that is reflective of the patient's state of recovery, a risk stratification score or number that is reflective of a probability or likelihood of a patient developing symptoms of a complication or risk of developing a condition that requires emergency treatment following a surgical procedure or combinations thereof. The risk and recovery scores are decision aids to clinicians similar to the standard practice of conducting patient assessments to determine what type of care, if needed, must be provided to the patients so that they can return to a healthy state.

In the present invention, a novel approach is presented to assess a patient during perioperative care, using systems and methods which can also provide not only assessment but also recommendations for post-surgical intervention planning, support, follow-up, patient compliance, recovery prediction and tracking, and potential treatment modifications. A user, relating to patients, such as physicians, medical professionals or the like, can receive predictive outcomes about a patient, based on the specific surgery or treatment or a plurality of multiple surgical procedures or measured physiological and biological data, historic patient medical data and records, and patient questionnaires. The method starts with obtaining input data from a patient, which can be obtained from a variety of available known sources. In certain preferred embodiments, the input data includes e.g., the multiparametric data inclusive of two channels of ECG and thoracic impedance, heart sounds near the apex of the heart, activity, and posture captured by a device such as the Nanowear® SimpleSense device (510 (k) number K212160) (Nanowear Inc. Brooklyn, N.Y.) combined with other data such as diet, information on medications taken and demographics data (e.g., age, gender, height, and weight). The SimpleSense™ device is a non-invasive, wearable, and portable medical device that uses cloth-based nanosensor technology (Rai, P. et al., Nano-bio-textile sensors with mobile wireless platform for wearable health monitoring of neurological and cardiovascular disorders, J. Electrochem. Soc., 161, B3116-B3150, (2013)). The garment was designed with an emphasis on ease of wearing and takes between 20 and 30 seconds for most subjects to put on.

In certain embodiments, the present invention is directed to a 3 step process: 1) obtaining input data; 2) selection of transformation for input data; and 3) translation of transformed input data into metrics.

### Step 1—Obtaining Input Data

Input data can be obtained by known methods including patient medical history data such as past diagnoses, test results for blood biomarkers, proteins, metabolites, and cholesterol and non-invasive medical devices that are intended to be used for collecting biomedical vital signs such as ECG, photoplethysmography, heart sound, activity, and calculate heart rate and respiration rate, physiological and biological data including, e.g., gathering inputs from the human body such as, but not limited to, electrical activity (ECG, EMG, EOG, EEG, GSR), mechanical action (Goniometric measurements of all joint angles, absolute inclination, and orientation of different parts of the body in multiple axes, photoplethysmographic measures at various sites on the body such as fingers, toes, ankle, foot, arms, thorax, neck, and forehead), sounds (heart sounds, lung sounds, gastrointestinal sounds, and joint sounds), impedance (acoustic, electromagnetic, and ultrasonic), blood oxygen levels, temperatures measured at different locations of the body inclusive of extremities, thorax, abdomen, and head, sweat biomarkers measured at various areas of the body such as lactate, pH, alcohol, nicotine, sodium, glucose, urea, chloride, discrete blood, interstitial fluid, cerebrospinal fluid (CSF) biomarker panels, and metabolic panels. Similarly, historic patient data includes e.g., height of the patient, a weight of the patient, a gender of the patient, an age of the patient, a medical history and physical examination records of the patient, a medical status of the patient, a body mass index (BMI) of the patient, an ethnicity of the patient, a medical prescription history of the patient, a medical prescription status of the patient, types of treatments and medications received by the patient, types of medical treatments for health issues and insurance or claims information previously received by the patient, diet information for the patient, psychological history of the patient, a genetic indicator of the patient, biomarkers of the patient, along with other Electronic Medical Records (EMR) information (EMR is a collection of all medical information associated with a patient historically and in the present time as data is captured and summarized as results for interpretation included in a patient's EMR). Preferred input data are measurements made from patients reflective of physiological condition like ECG, EMG, EEG, Phonocardiogram (PCG), activity and posture, sweat, blood and urine analysis results, and historical information on diagnosed conditions, past surgical interventions, levels of activity and absolute body posture with the inclination of the upper and lower body relative to the ground, as measured by an accelerometer, gyroscope, or an inertial measurement unit with the ability to measure movement in at least three axes inclusive of static measurement of the acceleration due to gravity, and history of medications. Other preferred input data are obtained from non-invasive medical devices that are intended to be used for collecting biomedical vital signs such as ECG, photoplethysmography, heart sound, activity, and calculate heart rate and respiration rate. Additional data can be obtained from patient responses to questionnaires that assess recovery such as Quality of Recovery questionnaires.

### Step 2—Selection of Transformation for Inputs

The selection of transformation for inputs of Step 2 is accomplished by a) conditioning the input data, b) feature engineering the input data and combinations thereof.

### a) Conditioning the Input Data

Data conditioning is the process of removing, attenuating, or diminishing the presence of patterns in the physiologic data that are irrelevant to the physiological phenomenon being measured. These irrelevant patterns in the data are also referred to as noise. The conditioning of data is done to essentially clean up the input data before feature engineering. The input data may contain known forms of noise or irrelevant information that it may be desirable to remove through conditioning before moving to feature engineering. Data conditioning includes methods such as filtering, trend removal in case there are gradual drifts in the measurement values due to the instrumentation used to perform the measurement, or other signal processing methods that increase the proportion of physiologically relevant data to the noise.

The methods of conditioning of the input data may include, but are not limited to, the following:
1) Transformations of the input data from the time domain to other domains such as frequency, wavelet using one of the wavelets (such as Coiflets, Daubechies, Meyer, Biorthogonal, Gaussian, Mexican hat, Morlet, Haar, Shannon, Complex Morlet) and/or cepstral analysis.
2) Application of filtering techniques such as homomorphic filtering, frequency-domain filtering of the time-domain, or transformed input signals to segment and extract quantitative or qualitative measures correlated with physiological factors in turn correlated to patient status assessment and trajectory towards recovery or complication following a surgical intervention. The determination of what to remove and what to retain is based on domain knowledge. For example, heart sound sensor or microphone recording the heart sounds on an individual may include conversation sounds from others nearby. These conversational sounds may be at a frequency range greater than the frequency range in which heart sounds manifest. A time-domain process filtering with the allowable frequency band set to the heart sound range can effectively remove the noise from the conversations of others in the vicinity.
3) Neural networks may additionally be used to apply transformations. Data may also be encoded or embedded into a higher dimensional space that preserves or differentially enhances or improves the signal-to-noise ratio so that the shared or mutual information between the input data and patient status assessments may be learned by a predictive machine learning model. This process is also referred to as basis expansion. An illustrative example is if the actual relationship between a measured input (X) and a value to be estimated (Y) in reality is A*X\hat{ }2+BX=Y, what is measured is only X, then higher dimensional embedding on X simply results in the input of X (actual measurements of X) and X\hat{ }2 (which is the variation of X in another dimension that is not linearly related to X) as the higher dimensional embedding of X so that an estimation of A and B can be begun with this new transformed data consisting of X and X\hat{ }2) that preserves or differentially enhances or improves the signal-to-noise ratio so that the shared or mutual information between the input data and patient status assessments may be learned by a predictive machine learning model.

The goal of data conditioning, with e.g., transformations or filters, is to remove to the maximal extent possible all variations within the data that are mathematically separable from the variations that are truly correlated to patient status assessments. To accomplish this task, the data in its raw measured form may be subjected to transformations from the time domain to other domains such as frequency, wavelet using one of the wavelets (such as Coiflets, Daubechies, Meyer, Biorthogonal, Gaussian, Mexican hat, Morlet, Haar, Shannon, Complex Morlet), or cepstral analysis. As the next step to differentially enhance the information that is correlated to patient status assessments relative to input data that is not correlated to patient status assessments, filtering techniques may be applied such as homomorphic filtering, frequency-domain filtering of the time-domain, or transformed input signals to segment and algorithmically measure quantitative or qualitative measures correlated with physiological factors in turn correlated to e.g., systolic and diastolic blood pressures or other physiologic parameters. Additionally, for the purpose of transforming the input data, neural networks may be used to apply transformations (All neural networks are generally a set of numerical computations consisting of multiplications, convolutions which is the same underlying computation as applying filtering in time domain, pairwise products known as dot or Hadamard products, or scaling (multiplying by a constant), or summation or applying various thresholding function to binarize or split into ranges any continuous number such as histograms, sigmoid functions, tan hyperbolic functions, Rectified linear unit (ReLU) or modifications thereof like leaky ReLU—these are also referred to as activation functions)—as the process of applying data to a neural network is essentially the same as applying a transfer function consisting of a sequence of mathematical operators (matrix multiplication, sum of all elements with or without multiplication by a scalar value referred to as weights, application of non-linear thresholding such as applying the input summation to a Logit function that limits the output between 0 and 1, and then applies a threshold for example 0.5 and all outputs above 0.5 are treated as 1 and below are treated as 0), that consist of a combination of mathematical operations and a selective transfer or ignoring of the output of parts of these computations referred to as skip connections). In the preferred embodiment, wavelet denoising, signal smoothening using running averages and Hilbert transform envelopes are used to condition the ECG and heart sounds data.

A pre-trained neural network is any neural network that is trained to perform either a classification (if the input is an image, the neural network output is whether there is a dog present in the image) or regression task (if the inputs are a geographic region and per capita income, then what is the estimate of house prices in that region). The pre-trained network is essentially not exposed to the input data chosen through the process of input selection described herein. However, the method or mechanism of extracting and emphasizing only certain features or aspects of the input to perform its function as trained originally, can be relevant to the estimation of patient status assessments. Such a pretrained neural network, can then be provided with the training data prepared for patient status assessments and can be trained or the internal terms or constants referred to as weights can be changed to better estimate patient status assessments from the chosen inputs. The idea is that the pre-trained neural network will already possess a mechanism to infer attributes from any input data. By re-training the networks, the transfer function implemented by the neural network is fine-tuned so that it will predict patient status assessments, instead of what it was originally trained to predict like whether there is a dog or not, and whether the region and per capita income is related to house prices in our example. Thus, the neural networks are not specifically designed for the purpose of predicting patient status assessments, but can be used from a pre-trained network after replacing the input layer of the neural network to match the dimensions of the input data chosen in the previously in this step.

In preferred embodiments, the input data is conditioned using an engineering system such as filtering methods to remove undesired noise or patterns that are irrelevant to the estimation of patient status assessment within the input data obtained. Appropriate filtering methods include finite impulse response filters, infinite impulse response filters, recursive filters, wavelet denoising, signal smoothening using running averages or autoregressive moving averages, and adaptive filters, ideal filters and optimal filters or combinations thereof.

After conditioning, the input data may need preparation. Preparation of data is the process of translating input data in a certain format or data type into a format that is compatible with Step 3 (translation of transformed inputs into metrics) which is the process of mathematically converting this data into values that are compatible with numerical methods. For example, if the mathematical conversion formula in the following step does not accept inputs in the form of text such as “Male” or “Female” and can only accept numbers or integers, then the data preparation step will perform the task of assigning numbers that represent “Male” and “Female” such as “Male” will be mapped or coded as 1 and “Female” will be mapped or coded as 2 prior to providing the data to the next step of mathematical conversion.

### b) Feature Engineering

Feature engineering is comprised of feature extraction, feature selection and combinations thereof. The process of feature extraction results in features and the process of feature selection results in a subset of features that are actually relevant to patient status assessments based on a measurement of the amount of mutual or shared information between the feature under evaluation (Mutual Information by definition relates two random variables. Mutual Information measures the dependence between the two from the information content perspective i.e., the measure of amount of information contained by a feature about the patient status assessments.) The two random variables in this case are a particular feature and the patient status assessments measured by a reference device such as a sphygmomanometer, simultaneously with measures of patient status assessments available from a reference device that is deemed as the ground truth measurement of patient status assessments associated with the same time instance of the input data that was used to compute or extract the feature under evaluation. The method(s) may consist of a rule-based approach to measure specific attributes of an input data type such as the amplitude associated with a periodically occurring pattern in the input data such as the R peak in the ECG or the characteristic heart sound amplitudes in the measured heart sounds, S1 (lub) and S2 (dubb). The features to be used are based on domain expertise and known associations between the targeted features and patient status assessments. Alternatively, an exhaustive evaluation of permutations of neural network architectures that may directly provide the transformation from the input data to features relevant for the estimation of patient status assessments.

i) Feature Extraction

Feature extraction is the process of measuring specific aspects within a physiological data set pertaining to a patient or summarizing the information present within a particular form of physiologic data. For example, in an ECG physiologic waveform, there are characteristic patterns referred as Q wave and T wave. The time elapsed between the occurrence of a Q wave and a T wave is a feature that is relevant or related to blood pressure at the corresponding time of measurement of the ECG. The process of listing all such possible features and the developing computer programs and algorithms (Makowski, D., Pham, T., Lau, Z. J., Brammer, J. C., Lespinasse, F., Pham, H., Schölzel, C., & Chen, S. A. (2021), NeuroKit2: A Python toolbox for neurophysiological signal processing, Behavior Research Methods, 53(4), 1689-1696. that can calculate such features is known as feature extraction. This approach of feature extraction is applicable to statistical processes that are categorized under machine learning methods but are alternatives, or complementary to machine learning, where initial assumptions on the relationship between a particular feature and patient status assessments are required. For example, a feature such as the time elapsed between the Q wave and T wave can be related to patient status assessments while recovering from a heart surgery procedure, then a feature can be defined as QT interval, so there is an implicit assumption that QT interval is related to patient status assessments for all patients which may not be true. Therefore, alternatively, the plurality of data as such without feature extraction and the accompanying required assumptions can be used as input to a neural network or deep neural network that is trained or optimized to estimate patient status assessments directly from the input data in an approach that is commonly referred to as end-to-end machine learning where no features need to be pre-defined and no assumptions are made regarding which components or portions of the input data are relevant to patient status assessments. The neural network training process will allow the neural network to infer the true relationship between the input data and the physical parameter, e.g., blood pressure.

Feature extraction involves techniques or methods such as, but not limited to, discrete Fourier and short-term Fourier transforms, discrete cosine transform, Autoregressive models (AR), Autoregressive moving average models (ARMA), classes of linear predictive coding models, cepstral analysis derived Mel-Frequency Cepstral Coefficients (MFCC), Kernel-based modeling, multiresolution analysis using discrete and continuous wavelet transformations, wavelet packet transformations and decompositions, empirical mode decompositions, power spectrum estimation using techniques such as Bartlett or Welch periodograms, Hilbert transforms, cross-spectral coherence that measures spectral coupling across different signal modalities, non-negative matrix factorization (NMF), ambiguity kernel functions, a subset of the layers from a pre-trained multilayer neural networks used as a transformation from input data into feature vectors in the feature space, unsupervised or supervised clustering methods like adaptive resonance-based neural networks, self-organizing maps, k-means clustering, k-nearest neighbors, Gaussian mixture models, and Naïve Bayes classifiers which group together similar feature sets (plurality of features extracted or plurality of statistically summarized inputs such as mean, standard deviation, or median) and assign group labels to each instance of a set of features. The preferred embodiment uses multiresolution analysis and signal decomposition using wavelet transforms to condition the heart sound data.

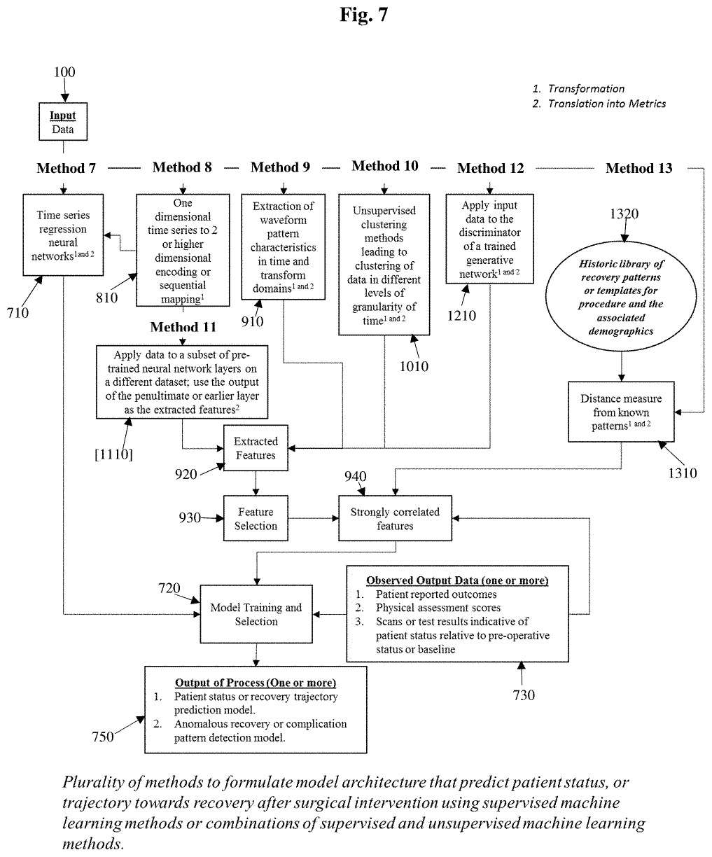

Feature extraction is generally always performed, with the exception of methods like Method 5 in FIG. 5 and Method 7 in FIG. 7. In Method 5, anomaly detection is performed on the input data directly to reveal statistically significant changes. Therefore, feature extraction is not explicitly needed to accomplish this step. In Method 7, the feature extraction is performed implicitly as part of the training of a time series regression neural network and therefore does not need a pre-specified feature extraction and selection to be performed on the input data.

ii) Feature Selection

Feature selection is only applicable in certain methods e.g., the supervised learning methods of FIG. 7 because supervised learning methods are applicable under conditions where there is a known outcome such as a patient status assessment available to be associated with a set of input data prior to the application of any methods. Feature selection is a step that is applicable only under such circumstances and not in the case of methods in FIG. 5 where outcomes are unavailable.

Feature selection involves two steps: 1) transformation and/or decomposition, and 2) selection. Transformation and/or decomposition is done to result in no change, or higher dimensionality of data, and the use of numerical methods to reduce dimensionality while preserving information and knowledge of variance. A transformation is the mathematical operation of manipulating a set of numbers that are representative of a state or measurement of a phenomenon using a series of mathematical operations. The manipulations may or may not be reversible in a mathematical sense. A reversible transformation is a mathematical method of manipulation of a data set that can be reversed or inverted and applied to the transformed data so that the original data set can be returned to. Such transformations are often used in filtering, where signal and noise are more easily separable by applying thresholds after being transformed. Wavelet denoising is a good example of this method—the transformation of input data in to the wavelet domain can be reversed or inverted so that the original data can be returned to without loss of information. Generally, transformations translate data from one domain to another. The present invention includes other domains to cover the use of neural networks to perform transformations like the use of a set of convolution neural networks with skip connections for example, which transform the data into an intermediate multi-dimensional space but not in any currently well-established definition of a domain (other than the traditional domains which are time frequency and wavelets).

Decomposition is the process of reducing a set of data (simplistically a matrix of numbers and more generally a high dimensional data set) into a set of constituent fundamental sets of numbers that can represent all the information present in the original set of data. Principle component analysis is a form of decomposition of a given set of numbers into independent principal components that are equivalent in terms of the information present in the original matrix. Decompositions are useful to determine how many independent fundamental components are truly needed to represent all the information in a given set of data (fewer allows for reducing the amount of data needed to be stored and also complexity of the algorithms needed because less important components are, in a sense, discarded. Transformation and/or decomposition involves techniques like box cox transformation, eigenvalue, and vector decomposition, principal component analysis (PCA), kernel PCA, truncated singular value decomposition, multidimensional scaling, and isometric mapping, and t-distributed stochastic neighbor embedding. The purpose of transformation and/or decomposition is to reveal how much pure information is present within the set of features that were extracted. The relevance of the features is the evaluated during selection. Transformation and/or decomposition of the features can be obtained by applying Principal Component Analysis, eigen value or vector decomposition, and box cox transformations to perform transformations to the features. These methods are typically performed so that a large set of features can be compressed into fewer set of features that represent most if not all of the variations of the features in terms of pure information contained in the features as assessed by statistical variance (essentially, more variation means more information). Feature selection evaluates whether the compressed form of information obtained through the transformation is relevant to patient status assessments with the goal of selecting features that are relevant to the assessment and eliminating or ignoring the others. Feature selection is done through the feature importance assessment which provides a rank ordered list of features based on how useful or relevant they are for the estimation of patient status assessments. The top N number of features is then selected based on the accuracy obtained with a model trained with N number of features as input, allowing for exhaustive training and evaluation of accuracy of models for all values of N from 1 to all features that were extracted. Methods appropriate for selection can also include measurement of mutual information using Kullback-Leibler convergence, minimum redundancy maximum relevance, impurity-based feature importance using random forest regression models, F-statistic or f-test, neighborhood component analysis, backward elimination, forward selection, permutation feature importance, factor analysis, and relief algorithm for regression, Preferred methods are minimum redundancy maximum relevance and impurity-based feature importance using random forest regression models.

Features can be extracted for e.g. every 60 seconds of data from a nanosensor device. If a particular 60 second duration of recording is very noisy and the features cannot be extracted from that segment, then imputation techniques can interpolate and estimate what the value of the features might be for that 60 second segment based on historical values for the feature, most recently calculated feature values, or values in the neighboring vicinity in terms of time. Thus, in instances where features cannot be computed or are computed erroneously due to underlying noise in the input data, imputation techniques like replacement of missing values with mean, median, or interpolation from neighboring values with linear, or non-linear methods such as cubic splines or higher order polynomials may be used. Once missing values are imputed, regression techniques such as Tree-based methods, support vector machines, logistic and linear regression with variable coefficients, Gaussian process models, and neural networks. Tree-based methods may include but are not limited to boosted trees, random forests, gradient boosting trees, extreme gradient boosting, AdaBoost, bagged trees, and an ensemble of tree-based models, which is a combination of any or multiple tree-based models. Support vector machines may include but are not limited to linear, cubic, quadratic, coarse, fine, or medium gaussian. Gaussian process regression models may include but are not limited to rational quadratic, squared exponential, exponential, Matern 5/2 kernels. Neural networks may include but are not limited to multilayer perceptrons, generalized regression neural networks, radial basis function networks, recurrent neural networks, long-short term memory networks, and convolution neural networks. The preferred embodiment is an ensemble average of regression models including k-nearest neighbor, Adaboost, gradient descent, support vector machines, and multilayer perceptrons.

Based on the data types available as input, an appropriate model and specific methods can be chosen from the list above or other suitable methods. In practice, the different methods can easily be tested to determine which gives the best results with the particular input data. For example, if a questionnaire is administered to cover assessments in multiple domains such as physiological, nociceptive, emotive, cognitive, and activities of daily life using an integer to indicate the answer to a multiple choice question, then simpler weighted summations could be used to create a score for a given patient. This score can then used to quantify the recovery status assessment and a threshold to distinguish recovered from not recovered can be established based on observations of the results of administering such a questionnaire in a large patient population where patients have undergone a specific type of surgery. Such a method is described in Royse et al., Development and Feasibility of a Scale to Assess Postoperative Recovery: The Post-operative Quality Recovery Scale, Anesthesiology, 2010; 113:892-905.

Although feature extraction and/or feature selection steps can be conducted outright in the process of the present invention, in certain methods when a time series regression neural network is used, the feature extraction and feature selection are not performed explicitly as separate steps but instead are implicit in the way a neural network is trained. The training process effects the neural network to learn the features that are relevant as part the neural network's parameters. Parameters are any constants or choices for variables required to be made or assumed as part of applying a mathematical or computational method. For example:—neural networks require initial values of weights for a given architecture—is a parameter. Gaussian process methods require an initial choice of kernel function which is one of a list of available options like radial basis function, matern kernel etc.

Generally, time-series data is a type of data that has a unique value associated with a series periodic or a periodic instance of time. For example, stock ticker for AAPL has a series of traded share prices for each instance in time over the past year. The value of the stock over time is a form of time-series data. Time series regression neural network is a type of neural network that is used to mathematically search and determine the causal relationship between a sequence of events (like a sequence of data representative of the time varying ECG data or one of the input data components used for patient status assessment) and an output like the estimated patient status assessment. They are specifically regression type networks because their output is continuously valued (i.e., can take the form of any continuous number—in this case a recovery score or a risk of complication score). Only an architecture is defined in this step (architectures are specifically defined by the number of layers, types of operations performed by the layers and how the output of a layer is transformed before applying it to the next layer, whether there are layers that are skipped and data from one layer forwarded to another layer further along in the architecture which can be viewed as a pipeline or stack, or certain outputs from one layer are discarded partially.

Step 3—Translation of Transformed Inputs into Metrics

Broadly all of the methods of the present invention, both unsupervised and supervised learning methods, will result in a metric, image, or a video representation that is reflective of the recovery status of the patient.

Supervised learning methods are applicable when a ground truth measurement of a quantity to be estimated is available, so the model can be trained to estimate that quantity better during training. This is called “supervised” because the outputs generated by the model can be actually supervised and iteratively make changes to the model parameters to improve performance.

Unsupervised methods on the other hand are more akin to data mining where measurements to guide the training process are not available. Instead, methods are used to define groups among instances of features that are more similar to each other than to instances of extracted features that are dissimilar. Unsupervised learning methods assume that all features are relevant and utilize all information without going through selection. A cluster is defined as a group of points that are similar to each other, more so than other groups of points. A cluster classification is the identity given to a cluster. For example, if there are 2 distinct clusters, then cluster classification for a new feature set that falls within the group defined for group 1 is 1.

The output can be obtained by a plurality of appropriate methods, e.g. applying an appropriate method to the selected features, such as support vector machines, linear regression, bagged trees, gradient boosting trees, extreme gradient boosting trees, Adaboost trees, random forests, k-nearest neighbors, gaussian process regression, or other kernel-based regression techniques, multilayer perceptron neural networks, recurrent neural networks, or convolution neural networks and combinations thereof. The preferred methods are a combination of gradient boosting trees, extreme gradient boosting trees, Adaboost trees, random forests, k-nearest neighbors, gaussian process regression, and support vector machines.

Step 3 searches for an optimal combination of the transformed or original data and a computational pipeline which is the sequence of computational operations needed from the input data to estimate a patient status assessment that includes data conditioning, preparation if needed, extraction of selected features and finally applying a computational model to estimate a recovery score or risk of complication score using these features extracted from input data or directly from input data. The pipelines may include normalizations, combination, and transformation that converts the inputs into a patient status assessment prediction that accurately matches simultaneously made reference assessments from a standard of care assessment tool such as Quality of Recovery questionnaire (QoR-15) (Stark P. A., Myles P. S., Burke J. A., Development and psychometric evaluation of a postoperative quality of recovery score: the QoR-15. Anesthesiology. 2013; 118: 1332-1340).

The purpose of normalization is to standardize and restrict the range of the input data from reference sources that are used for model assessment and improvements such as QoR-15. The purpose of combination is to have a method of determining the computational method needed to determine the extent to which the model and signals need to be improved for a model that is already estimating patient status assessments. The transformation converts the outputs of the combination step to a value to improve or correct the model, or a signal that can improve or correct the signal, respectively.

In the present invention, the methods can include using scores or scales to assess the patterns of change. Monitoring and assessment of patient status as indicated for surgical intervention may begin at and extend to any of the stages of perioperative care inclusive of preoperative, intraoperative, or postoperative. Monitoring can include periodic assessment of cognitive, psychological, behavioral, and physiological changes and trending of these assessments over time. Together, cognitive, psychological, behavioral, and patient history and etiology of the condition requiring surgical intervention, provide an instantaneous assessment of the holistic state of the patient. As such, methods to track the evolution of this holistic state will provide information on the evolution of patient status towards recovery or exacerbation. In addition, an instantaneous assessment and historic assessments can be used to predict the trajectory of patient status at a future point in time along with an associated confidence in the prediction. In preferred embodiments, the associated confidence is e.g., 75%-95%. As more patients are assessed, a historic library of recovery patterns or templates or procedures and the associated demographics is first created and then continuously added to, such that the associated confidence improves over time as more and more historical data is obtained. The historic library is relevant to patterns or trends centered in time around an event such as a surgery. Predictions can further be used as a means of stratifying patients based on the risk of worsening condition, sub-par rate of recovery, or similarity to known patterns of expected recovery under similar conditions of patient type, procedure type, and history, or demographics. Such a risk stratification could be used at the level of a practice, hospital, or care provider, or across several patients to provide clinicians with insights on which patient will require more attention and care.

The methodology for development of such a system for continuous assessment of patient status may consist of three steps—Selection of inputs, search for a transformation of the inputs to emphasize the information that is mutual or shared between the inputs and the evolution of patient status over time, and methods to reduce the information into quantifiable metrics that may be presented as a numeric, symbolic, image or video so that it can be used or consumed by clinicians as decision support for patient care.

Model training is the process of improving the accuracy of a model represented mathematically as a mathematical function or operation that associates a set of chosen inputs with the desired outcome that is a predicted assessment. As each set of input data is presented, the model's mathematical calculation is performed to get an estimated output. This estimated output is compared to the ground truth value measured using a reference gold standard method, and an error also known as cost is determined using a pre-defined cost function. Some examples of cost functions are mean squared error, root mean squared error, mean absolute error, r squared, and log-loss. Depending on the value of cost determined by the difference between the estimated value and the expected value, the parameters or coefficients that are part of the model are updated following certain rules. Some examples of rules are gradient descent, root mean square propagation, adaptive resonance, and Adam optimizers. These methods are standard among neural network training Other methods in machine learning such as Support vector machines, Gaussian process regression, k nearest neighbors, Adaboost, extreme gradient descent all have algorithms for training that are unique to the methods and also require pre-specification of hyperparameters for the models when they are created (created essentially means defining a starting point or initialization point and a basic structure in terms of mathematical computational units used, some examples for gaussian process regression are choice of pre-defined kernel functions—5/2 matern, white noise, and radial basis function. Similarly for support vector machines some hyperparameters are—regularization parameter, kernel functions, and maximum iterations) for training Model training results in multiple models which can be one of such regression models. Model selection is the process of choosing the model that results in the lowest error in terms of difference between the estimated patient status assessment score and a known patient assessment score. This error maybe calculated as the mean absolute difference, mean squared difference, standard deviation of errors, or Huber loss.

In preferred methods to obtain the Output of the Process, several different types of models are trained on the training data and their performance is evaluated on an independent test data set that is collected from a different group of subjects that the data from the training set. Depending on the error performance measured by established metrics such as mean squared error, mean absolute error, or r squared, the best performing model is selected. So, the output is the best performing model that includes all steps that led to the definition of that model inclusive of choice of input data, conditioning and preparation of data, transformation and/or decomposition of data, and finally the machine learning algorithm or combinations of machine learning algorithms that were used in the model. The input data of Step 1 are applied to models after data conditioning which may involve filtering in time, frequency, wavelet, or other domains using a reversible transformation. Preferably, this involves filtering in time domain and wavelet domains. The choice of the data conditioning methods is dependent on the input data type and domain knowledge regarding what is a signal and what is noise or irrelevant information in the input data. For example, frequency domain filtering is preferred when the input data is ECG and the noise source is power line interference. The inputs with or without conditioning may be retained in time-series format to be supplied to a class of neural networks or deep learning architectures that have output layers comprised of linear transformations that sum and scale the output from the previous layer commonly referred to as regression output layers, that support continuous-valued regression outputs (with deep learning architectures being a subset of neural networks that have several computational layers that categorizes them as deep, as opposed to shallow neural networks which have fewer computational layers). The output layer of a neural network produces a regression output, i.e., continuous valued output. The output layer is defined by either a single neuron in the case of separate models for the assessment, e.g., systolic and diastolic blood pressures or other physical parameters, or two neurons if e.g., systolic and diastolic pressures and/or other physical parameters are estimated using separate models. Each neuron multiplies the weights (W1, W2, W3, and so on) assigned to each connection from the previous layers, sums the values, and finally applies a linear scaling or multiplicative factor to the sum to calculate an output.

The computational method used for the perioperative assessment can be developed using a set of data collected from a large patient population. Preferably, the patient population has at least about 50 patients, and, more preferably, at least 80 patients. A computational model may be developed using Method 14 in FIG. 5 by selecting the input data as all the data from Nanowear® SimpleSense™ device and demographic data such as age, gender, height, weight, and type of surgical procedure with information on the site of the surgery on the body. The input data can be conditioned using finite impulse response filters and Butterworth filters for ECG, and wavelet denoising using ‘coiflet5’ wavelet for heart sounds. Signal averaging could further be used to reduce noise as part of signal conditioning. The features listed in Table 1 can be extracted using signal processing methods. For example, algorithms defined to specifically discern patterns such as to find peaks in ECG waveform such as the R peak using an algorithm (such as described by J. Pan et al., A Real-Time QRS Detection Algorithm,” in IEEE Transactions on Biomedical Engineering, Vol. BME-32, no. 3, pp. 230-236, March 1985), and the time of its occurrence and similarly, peaks in the heart sound loudness such as the characteristic S1 and S2 heart sounds using an algorithm as described by Kumar P. S. et al., Multiparametric cloth-based wearable, SimpleSense, estimates blood pressure, Sci Rep 12, 13059 (2022). Once the features of Table 1 are extracted, they are combined with the simultaneously acquired measurements from a reference device such as a sphygmomanometer to create the training data set. After the creation of the trained data set, model training and selection is performed. Model training is the process of improving the accuracy of a model represented mathematically as a mathematical function or operation that associates a set of chosen inputs with the desired outcome that is a predicted assessment. As each set of input data is presented, the model's mathematical calculation is performed to get an estimated output. This estimated output is compared to the ground truth value measured using a reference gold standard method, and an error also known as cost is determined using a pre-defined cost function. Some examples of cost functions are mean squared error, root mean squared error, mean absolute error, r squared, and log-loss. Depending on the value of cost determined by the difference between the estimated value and the expected value, the parameters or coefficients that are part of the model are updated following certain rules. Some examples of rules are gradient descent, root mean square propagation, adaptive resonance, and Adam optimizers. These methods are standard among neural network training Other methods in machine learning such as Support vector machines, Gaussian process regression, k nearest neighbors, Adaboost, extreme gradient descent all have algorithms for training that are unique to the methods and also require pre-specification of hyperparameters for the models when they are created (created essentially means defining a starting point or initialization point and a basic structure in terms of mathematical computational units used, some examples for gaussian process regression are choice of pre-defined kernel functions—5/2 matern, white noise, and radial basis function. Similarly for support vector machines some hyperparameters are—regularization parameter, kernel functions, and maximum iterations) for training

### Step 1—Selection of Inputs

This step involves selecting and collecting a set of measurements or inputs that have information that is mutually shared with the predicted quantities, which may include metrics such as risk stratification by means of a hazard ratio or index, or a recovery percentage score indicative of change and trajectory of change in a patient's status around an index event which involves a surgical intervention. These inputs may be time-series signals, i.e., signals measured periodically such as ECG, heart sounds, pulse oxygenation, pulse rate, activity levels, blood flow pulse wave velocity measured at different regions of the body such as wrist, foot, forearm, or near the heart, or measurements made with preserved information of time of measurements such as heart rate, blood biomarkers indicative of disease status, chronic diseases, or metabolic abnormalities. In addition, data may include a patient's history and physical examination data, claims information that could be reflective of past medical problems or events, notes from electronic medical records, patient-reported symptoms and notes, and demographics data such as age, gender, height, and weight.

### Step 2—Selection of Transformation for Inputs

This step is the preparation of the input signals or discrete data points which may also be referred to as feature extraction and feature engineering. The methods of preparation may include, but are not limited to the following—transformations of the input data from the time domain to other domains such as frequency, wavelet using one of the wavelets (such as Coiflets, Daubechies, Meyer, Biorthogonal, Gaussian, Mexican hat, Morlet, Haar, Shannon, Complex Morlet), cepstral analysis and application of filtering techniques such as homomorphic filtering, frequency-domain filtering, or transformed input signals to segment and extract quantitative or qualitative measures correlated with physiological factors in turn correlated to patient status. Additionally, for the purpose of transforming the input data, neural networks may be used to apply transformations. All neural networks are generally a set of numerical computations consisting of multiplications, convolutions which is the same underlying computation as applying filtering in time domain, pairwise products known as dot or Hadamard products, or scaling (multiplying by a constant), or summation or applying various thresholding function to binarize or split into ranges any continuous number such as histograms, sigmoid functions, tan hyperbolic functions, Rectified linear unit (ReLU) or modifications thereof like leaky ReLU—these are also referred to as activation functions. As the process of applying data to a neural network is essentially the same as applying a transfer function consisting of a sequence of mathematical operators (matrix multiplication, sum of all elements with or without multiplication by a scalar value referred to as weights, application of non-linear thresholding such as applying the input summation to a Logit function that limits the output between 0 and 1, and then applies a threshold for example 0.5 and all outputs above 0.5 are treated as 1 and below are treated as 0), the may consist of a combination of these mathematical operations and a selective transfer or ignoring of the output of parts of these computations referred to as skip connections).

Data conditioning is the process of removing, attenuating, or diminishing the presence of patterns in the physiologic data that are irrelevant to the physiological phenomenon being observed through measurement. These irrelevant patterns in the data are also referred to as noise. Data conditioning includes methods such as filtering, trend removal in case there are gradual drifts in the measurement values due to the instrumentation used to perform the measurement, or other signal processing methods that increase the proportion of physiologically relevant data to the noise. The goal of data conditioning is to remove to the maximal extent possible all variations within the data that are mathematically separable from the variations that are truly correlated to the assessment. To accomplish this task, the data in its raw measured form may be subjected to transformations from the time domain to other domains such as frequency, wavelet using one of the wavelets (such as Coiflets, Daubechies, Meyer, Biorthogonal, Gaussian, Mexican hat, Morlet, Haar, Shannon, Complex Morlet), or cepstral analysis. As the next step to differentially enhance the information that is correlated to the assessment relative to input data that is not correlated to the assessment, filtering techniques may be applied such as homomorphic filtering, frequency-domain filtering of the time-domain, or transformed input signals to segment and algorithmically measure quantitative or qualitative measures correlated with physiological factors in turn correlated to physical parameters such as systolic and diastolic blood pressures. Additionally, for the purpose of transforming the input data, neural networks may be used to apply transformations (All neural networks are generally a set of numerical computations consisting of multiplications, convolutions which is the same underlying computation as applying filtering in time domain, pairwise products known as dot or Hadamard products, or scaling (multiplying by a constant), or summation or applying various thresholding function to binarize or split into ranges any continuous number such as histograms, sigmoid functions, tan hyperbolic functions, Rectified linear unit (ReLU) or modifications thereof like leaky ReLU—these are also referred to as activation functions)—since the process of applying data to a neural network is essentially the same as applying a transfer function consisting of a sequence of mathematical operators (matrix multiplication, sum of all elements with or without multiplication by a scalar value referred to as weights, application of non-linear thresholding such as applying the input summation to a Logit function that limits the output between 0 and 1, and then applies a threshold for example 0.5 and all outputs above 0.5 are treated as 1 and below are treated as 0), the may consist of a combination of these mathematical operations and a selective transfer or ignoring of the output of parts of these computations referred to as skip connections).

Conditioning methods do not perform rejection of data based on potentially incorrect methods of acquiring input data. The input data can be corrected by e.g., rule-based rejection of data that is known to be noisy or unusable for the patient status assessments due to the presence of noise that makes it impossible to observe the signals or in cases where data was captured under circumstances or conditions that would lead to bias in the information carried by the input that is correlated to the assessment. Correction methods can also include an adaptive filtering method such as a recursive least squares filter that can differentially remove a noise with a known pattern while preserving the signal. An example of such a method is the use of accelerometer data that is reflective of movements, embedded in a wearable device, as a measurement of noise when it is known that movement specifically interferes with the measurement of ECG signals. A correction method can be used to remove data collected by a measurement device used in a manner that does not produce the correct measurement, wherein the correction method accounts for data quality and confounders and is selected from the group consisting of thresholding techniques for level of movement, adaptive filtering techniques for remediation such as recursive least squares filtering and combinations thereof. For example, if collecting data only when the patient or wearer of a medical device is stationary is a requirement for a particular measurement device, provided that the device possesses the means to measure movement, as present in Nanowear® SimpleSense™ device by way of an accelerometer sensor, the data collected during movements can be corrected by rejecting or ignoring the data from further processing. Alternatively, remediation of the data by using the movement data to remove any patterns in the measurement that are caused solely due to movements. The correction method to account for the data quality and confounders such as thresholding techniques for level of movement, such as an amplitude threshold, frequency content threshold, or adaptive thresholds, and adaptive filtering techniques for remediation such as recursive least squares filtering.

Data may also be encoded or embedded into a higher dimensional space (this process is also referred to as basis expansion, an illustrative example is if the actual relationship between a measured input (X) and a value to be estimated (Y) in reality is A*X\hat{ }2+BX=Y. Only X is measured, then higher dimensional embedding on X simply results in the input of X (actual measurements of X) and X\hat{ }2 (which is the variation of X in another dimension that is not linearly related to X) as the higher dimensional embedding of X so that an estimation of A and B can be begun with this new transformed data consisting of X and X\hat{ }2) that preserves or differentially enhances or improves the signal-to-noise ratio so that the shared or mutual information between the input data and the assessment may be learned by a predictive machine learning model.

A preferred embodiment uses the steps following Method 1 which uses clustering methods as a transformation and then a mathematical model to convert the cluster memberships into a quantifiable metric that is representative of change in patient status. For example, assuming input data is available from the day of the surgery for a patient from Nanowear® SimpleSense™ data, the change quantified by changes in the overall cluster memberships using Method 1 is indicative of change in patient status. In this condition, no change reflects no recovery and any change may reflect a path towards recovery.

In case of instances where features cannot be computed or are computed erroneously due to underlying noise in the input data, imputation techniques like replacement of missing values with mean, median, or interpolation from neighboring values with linear, or non-linear methods such as cubic splines or higher order polynomials may be used. Erroneous computations can be detectable algorithmically as features outside a plausible range. For example, if a respiration rate of 500 breaths per minute is returned by an algorithm used to extract respiration rate, that instance of the feature can be determined as erroneous because it is not reasonably possible for patients to be breathing in and out 500 times in a minute). Features that change more rapidly in time will require higher order polynomials to fit the variations well and provide better estimates of what the value of that feature should have been at an instance when a feature could not be determined.

The inputs can be subjected to dimensionality reduction through techniques such as box cox transformation, eigenvalue, and vector decomposition, principal component analysis (PCA), backward elimination, forward selection, random forests impurity-based importance, permutation feature importance, factor analysis, linear discriminant analysis, truncated singular value decomposition, kernel PCA, t-distributed stochastic neighbor embedding, multidimensional scaling, and isometric mapping.

Preferably, data should be collected before surgery so that a baseline from before the surgery is obtained. However, the methods of the present invention are not restricted in this manner and the methods can be applied at any time perioperatively.

Step 3—Translation of Transformed Inputs into Metrics

This step involves a methodology to convert the computed features or structured data into a quantitative or qualitative metric that can be used to reflect a patient's recovery status or trajectory directly, or relative to other patient(s). Models may be developed in two broad approaches in machine learning that fall under supervised or unsupervised machine learning. For this description, supervised learning approaches are defined as methods that are applicable to problems wherein observations of the expected outputs and the corresponding set of inputs are available prior to the development of the model's architecture. The outputs following different methods depend on the methods applied and their mathematical constraints in terms of number of dimensions of length of a numeric vector or array that is possible as an output for each method. In Methods 1 and 2 of FIG. 5, the output is mathematically limited to being a single number (integer or continuous decimal), so it can be reflective of risk of complication or its complementary assessment, the chance of recovery. For Method 3, the output is a count of the number of deviations or the extent of deviation from a known pattern of recovery which indicates that it could be a quantity that could be reported as yes deviated or not at continuously measured instants of time.

Burden is defined as the number of deviations detected over the total number of assessments or measurements made. Numeric risk stratification is a scaled version of the burden so that the range of values may be limited numerically such as 0-100 patient recovery score is complementary to the numeric risk stratification in that it represents the complementary scenario i.e., the chance of recovery. For Method 4 (discussed in depth below), the output is multidimensional so that there is information that can be represented in more than one number. It can be represented as images in binary or grey scale for a 2-dimensional output in binary, or continuous numbers, or color images with 3-dimensional output with a minimum of 3 dimensions and 3 basis in the third dimension (to indicate color, red, green, and blue), or color images that can be associated with each instant of time so that they may be rendered as videos which are a sequence of images. For example, in Method 5 (discussed in depth below), the output is a count of number of anomalies, so it can either be a ratio of the number of occurrences of anomalies over total number of instances over which observations were made defined as burden, or it can be burden over a specified period of time like 1 day to 180 days or 365 days. For Method 6 (discussed in depth below), as distance measures are not limited in terms of number of dimensions, the outputs are a sum of all outputs possible from other methods. A preferred embodiment is that of Method 14 (discussed in depth below), which results in a single metric that is the simplest.

Unsupervised learning approaches are defined as methods that are applicable to problems wherein observations of the outputs of the model under development are unavailable. Unsupervised approaches may involve clustering methods such as but not limited to k-means clustering, hierarchical clustering, or a neural network-based method such as self-organized maps, k-nearest neighbors' methods, support vector machines, gaussian mixture models, naïve Bayes clustering, Agglomerative Clustering, balanced iterative reducing and clustering using hierarchies (BIRCH), Density-Based Spatial Clustering of Applications with Noise (DBSCAN), Mini-Batch K-means, Ordering Points To Identify Cluster Structure (OPTICS), spectral clustering, or affinity propagation. The optimal number of clusters may be chosen using methods such as the elbow point of the plot of number of clusters vs total within cluster sum of squares, gap statistic method, the silhouette method, or sum of squares method. There is a plurality of approaches to unsupervised learning to translate transformed inputs into metrics. Preferred methods are k-means clustering and hierarchical clustering as applied in Method 14.

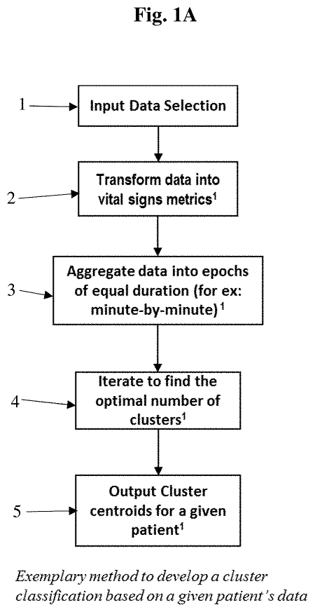

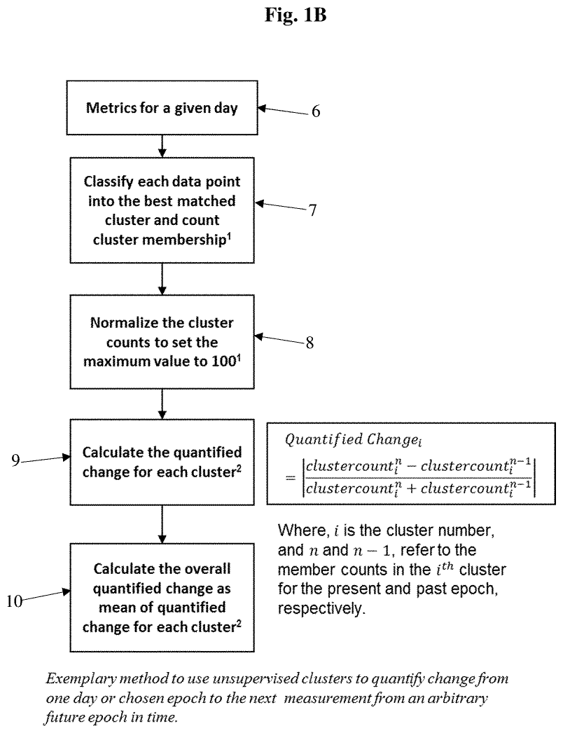

FIG. 1(a) (which correlates with FIG. 5, Method 1, step [110]) shows an exemplary method to develop a cluster classification based on a given patient's data. FIG. 1(a) starts with the selection of input data [1] which is then transformed into vital signs metrics [2]. In the next step, the data is aggregated into epochs of equal duration (for example, minute by minute) [3] after which the data is iterated to find the optimal number of clusters [4]. Finally, output cluster centroids are obtained for a given patient [5] (A centroid is a vector that is representative of a cluster. The centroid is the multi-dimensional average of all the points within a cluster.)

FIG. 1(b) (which correlates with FIG. 5, Method 1, step [120]) shows an exemplary method to use unsupervised clusters to quantify change from one day or chosen epoch to the next measurement from an arbitrary future epoch in time. In FIG. 1(a), the method starts with obtaining metrics for a given day [6], followed by classification of each data point into the best matched cluster and count cluster membership [7]. Next the cluster counts are normalized to set the maximum value to 100 [8], after which the quantified change for each cluster is calculated [9] using the following equation:

$$Quantified change i = \frac{{clustercount}_{i}^{n} - {clustercout}_{i}^{n - 1}}{{clustercount}_{i}^{n} + {clustercout}_{i}^{n - 1}}$$

where i is the cluster number, and n and n−1 refer to the member counts in the i^{th} cluster for the present and past epoch respectively.
   Finally, overall quantified change is calculated as a mean of quantified change for each cluster [10].

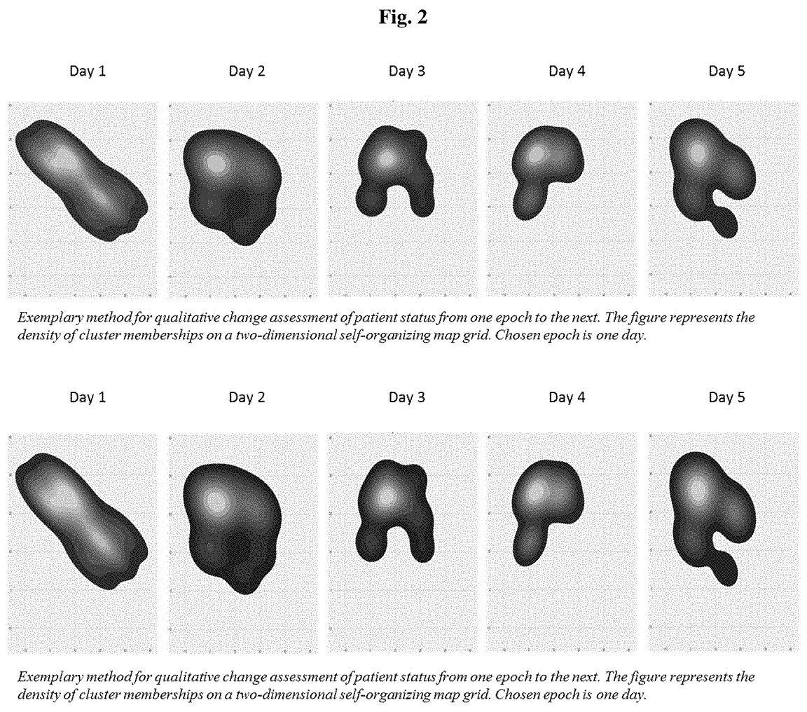

FIG. 2 shows an exemplary method for qualitative change assessment of patient status from one epoch to the next. The figure represents the density of cluster memberships on a two-dimensional self-organizing map grid, where the chosen epoch is one day. The differences in the appearance signify changes from day to day for a patient being monitored post-surgery. As several patients are observed over time, similar patterns of changes may emerge for specific demographic of patients undergoing specific surgeries. For example, male patients in the age range of 60- and 65-years undergoing Coronary Artery Bypass Graft (CABG) may have a pattern of change from day to day that is normal and any deviations from that pattern may be concerning to the caregivers. The patterns are also provided in color and grey scale to emphasize that the outputs for greyscale are numerically a 2-dimensional matrix drawn as an image with each value in the matrix representing a grey-scale number, whereas the color image is a 3-dimensional matrix that is essentially a stack of 3, 2-dimensional matrices representing red, green, and blue so that together we have a representation of color. For visual interpretation, colors are preferred because more subtle changes can be expressed and fundamentally color images convey more information than greyscale images.

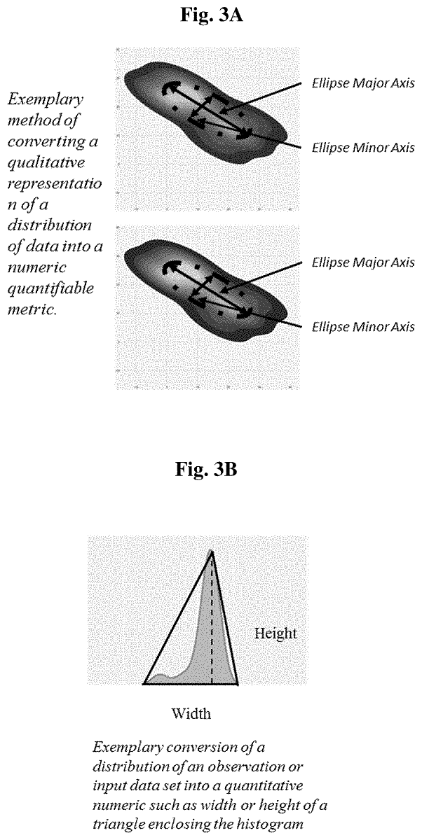

FIG. 3(a) shows an exemplary method of converting a qualitative representation of a distribution data into a numeric quantifiable metric. The qualitative representation in this case reveals an elliptical shape. An ellipse is geometrically uniquely defined by its major and minor axes. In this example, these two measurements could convey the meaning of an ellipse without having to display an ellipse. The major and minor axes can be extracted using a simple algorithm such as applying an edge detector or a 2-dimensional high pass filter and then finding the pixels associated with edges that are farther apart to represent the major axis and find the length of the segment perpendicular to this major axis that intersects the pixels that represent the edges. FIG. 3(b) shows an exemplary conversion of a distribution of an observation or input data set into a quantitative numeric such as width or height of a triangle enclosing the histogram. For example, an algorithm to find the smallest bounding triangle could be based on the algorithm described by Pârvu, O et al. (Pârvu, O., Gilbert, D, Implementation of linear minimum area enclosing triangle algorithm, Comp. Appl. Math. 35, 423-438 (2016).

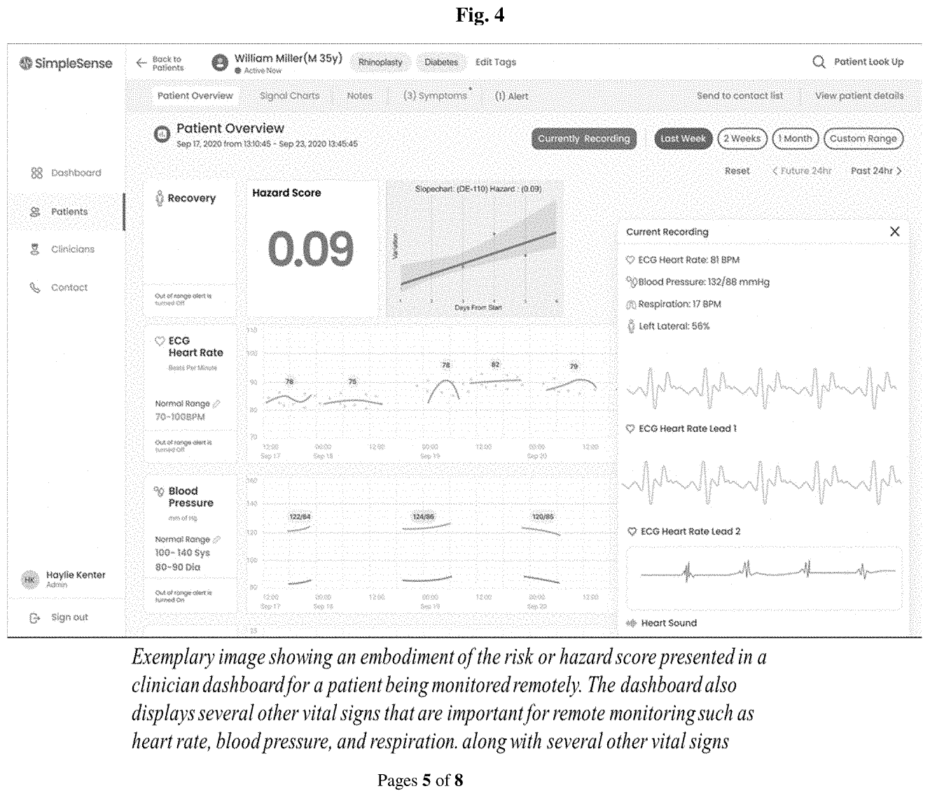

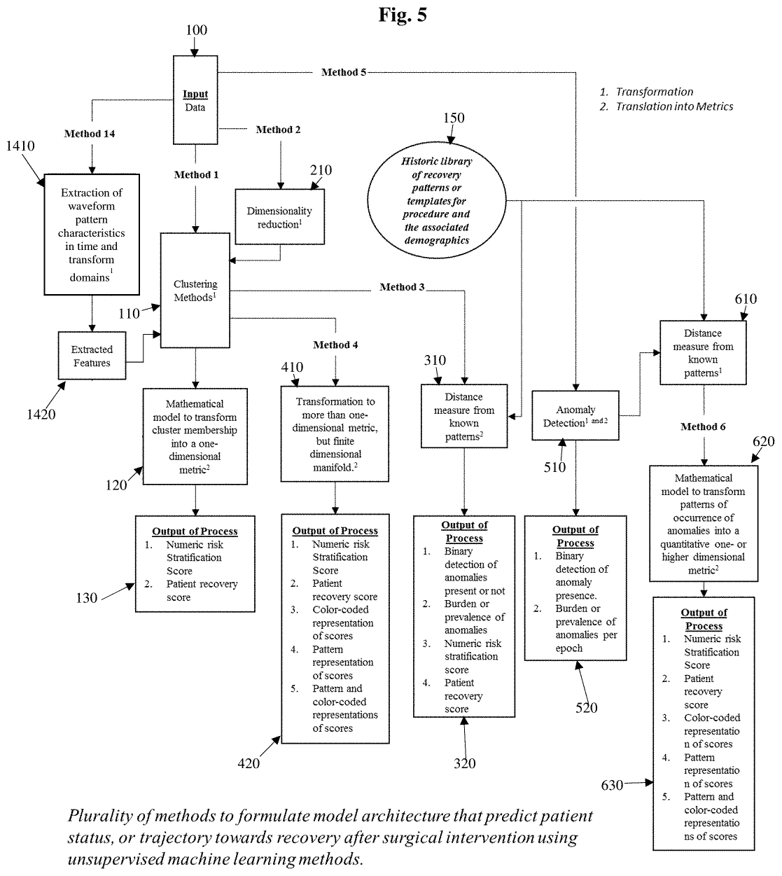

In FIG. 4, an exemplary clinician dashboard for a patient being monitored remotely is shown which displays vital signs that are important for remote monitoring such as heart rate, blood pressure, and respiration along with several other vital signs. Included in the dashboard is an embodiment of the risk or hazard score which shows a 0.09 risk which indicates the likelihood on a scale of 0 to 1 of not recovering. This hazard score is calculated using the method described in FIG. 1 using Nanowear® SimpleSense™ data obtained from a patient after a surgical event, with monitoring continuing for 7 days following the surgery.

FIG. 5 shows a number of exemplary methods (Methods 1-6 and 14) to formulate model architecture that predict patient status, or trajectory towards recovery after surgical intervention using unsupervised machine learning methods. Method 1 is preferred from the perspective of a purely information driven approach where there are no steps applied to modify or restrict the conversion process from input data to a score reflective of patient status. This is a desirable approach because there is no risk of bias due to the application of rules to modify the data based on any a priori assumptions on how the data relates to recovery and patient status.

In Method 1, input data [100] is obtained and then subjected to clustering methods [110], after which the output of the clustering methods (the cluster membership) is transformed by mathematical model, an example of such as model is—

$$Quantified {Change}_{i} = \vert \frac{{clustercount}_{i}^{n} - {clustercout}_{i}^{n - 1}}{{clustercount}_{i}^{n} + {clustercout}_{i}^{n - 1}} \vert$$

where, i is the cluster number, and n and n−1, refer to the member counts in the cluster for the current and previous epoch (An epoch is the duration of time over which the input data spans), respectively, into a one-dimensional metric [120]. Alternatively, examples of appropriate mathematical models include principal component analysis-based dimensionality reduction to a single dimension and then a statistical summarization of the principal component using mean, standard deviation, or variance followed by normalization and scaling such as min-max scaling to restrict the range of the metric to a scale of 0 to 1 or 0 to 100, The mathematical model may also be the output of an algorithm applied to summarize images into metrics such as the examples in FIG. 3. Additionally, the exemplary mathematical model described in FIG. 1 for quantified change is a preferred embodiment using the mathematical model for Quantified Change. Finally, the output of the process, which is the result of the mathematical formula or model [120], is obtained [130] (following the example the obtained output is the quantified change per epoch) resulting in a numeric risk stratification score which is indicative of the likelihood of a complication leading to recovery or worsening of the patient's health following a surgery and a patient recovery score, complementary to the risk stratification score indicates the likelihood of recovery to baseline conditions after surgery. The scores do not provide an assessment of health. They provide an assessment of change after a surgery, e.g., an assessment of change in the physiological, behavioral, and cognitive status of patients. The mathematical model that serves as an example is the following for the determination of change:

$$Quantified {Change}_{i} = \vert \frac{{clustercount}_{i}^{n} - {clustercout}_{i}^{n - 1}}{{clustercount}_{i}^{n} + {clustercout}_{i}^{n - 1}} \vert$$

where, i is the cluster number, and n and n−1, refer to the member counts in the ith cluster for the current and previous epoch (An epoch is the duration of time over which the input data spans), respectively, into a one-dimensional metric.

The risk score or its complementary recovery score will provide clinicians with insights that could help them plan interventions if needed or follow up with patients to guide them further on a quicker path to recovery. Alternatively, or concurrently, clinicians could triage among patients and determine which patients, among all patients that have undergone surgery, require more attention. The risk scores or recovery scores can be presented with a confidence level to indicate the likelihood of a false positive or a false indication of elevated risk of complication. For example, if the confidence level reported were 70% as opposed to 90% for the same hypothetical risk score of 0.9, in the case of 70% confidence, clinicians may try to look for additional information using other standard of care tools to confirm that the patient may be worsening. On the other hand, with 90% confidence clinicians may plan an intervention immediately to avoid complications for the patient. In the first method, the input data can be clustered using an unsupervised method described in the methods for Step 3. The number of data points classified in each cluster can then be subjected to a mathematical transformation that results in a one-dimensional metric. This metric may be associated with a risk stratification score or a patient recovery score that is an unbiased estimator of change in a patient's status from one chosen epoch of time to the next.

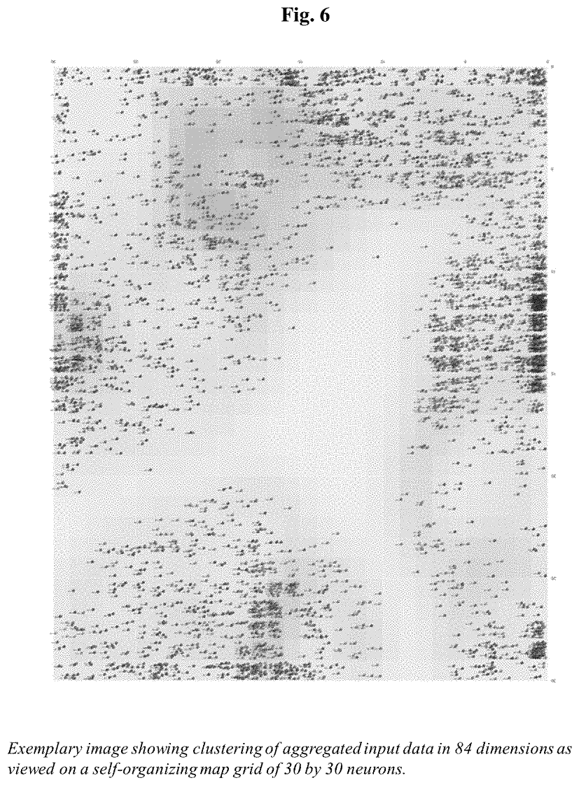

FIG. 6 is an exemplary image showing clustering of aggregated input data in a plurality of dimensions as viewed on a self-organizing map grid of 30 by 30 neurons. A self-organizing map is an unsupervised machine learning technique used to create a representation of a higher dimensional data set while preserving the information on the similarity of data points such that similar data points appear close to each other. For example, in case of the exemplary feature set with 85 features extracted in minute-level granularity observations as listed in Table 1, the data could be represented as clusters of observations that are similar. FIG. 6 is such a map-like visualization of clusters.

In Method 2, the input data [100] is obtained and then subjected to dimensionality reduction [210], which is a form of compression of the data so that fewer variables can be introduced that can explain the information present in the data with minimal to no loss. For example, principal component analysis represents high dimensional information in the form of principle components which can be selected based on their variance to choose a minimum number of principal components needed to explain the variance in the original high dimensional data set. Afterward, the remaining steps of Method 1 are followed: clustering methods [110], followed by transformation to a one-dimensional metric [120] to achieve the output of process [130]. The reduced dimensional data can be clustered, and a mathematical transformation can be used to transform the number of data points classified in each cluster into a one-dimensional metric. The transformation may be a linear or non-linear transformation that can preserve information on high-dimensional variations after transformation. The transformed output can further be scaled to a limited range for example from 0 to 1 or 0 to 100. Such values can be trended from one epoch to the next.

In Method 3, the input data [100] is obtained and subjected to dimensionality reduction [210] of Method 2 and then the clustering methods [110] of Method 1, after which the distance is measured from known patterns is calculated [310] using the cluster membership data compared with additional data obtained from an historic library of recovery patterns or templates for the procedure and the associated demographics. Each known pattern has a set of observations of features extracted from the same input data as used for Method 2. The distance of the current observation i.e., an observation in time of the input data from a patient for whom the recovery score or risk score is being computed, from a list of observations that are representative of typical patients undergoing the same surgery and belonging to the same demographic group, is a measure of how similar the current observations are to known observations in the past or historical observations. The mathematical functions to compute distance may be any one or combination of Euclidean, Manhattan, Mahalanobis, Minkowski, Hamming, and cosine distance.

The historic library can be accumulated by using devices to monitor patients undergoing surgeries such as the Nanowear® SimpleSense™ device and the patient's historical medical records data. This library can be accumulated over-time to capture different patient population undergoing surgeries. In preferred embodiments, the number of patients tested per surgical procedure is at least 50 patients. In certain embodiments, up to 10,000 or more patients can be tested. The output of the process [320] is then obtained as the mathematical operation of measuring the distance using a distance function [310]. The distance functions may be any one or combination of Euclidean, Manhattan, Mahalanobis, Minkowski, Hamming, and cosine distance. The distance from the pattern can be used to discern similarity or deviation from an expected pattern associated with a particular procedure type and demographic of patients. As a third method, the input data with or without dimensionality reduction can be compared to a known pattern of recovery or recovery trajectory to find a distance from the pattern in one or more dimensions.

In Method 4, the input data [100] is obtained and subjected to the reduction [210] of Method 2 and then the clustering methods [110] of Method 1, after which the cluster membership is transformed to more than one-dimensional metric, but finite dimensional manifold which is a representation of the points on a topological surface with more than one dimension [410]. Essentially, this is a means to visualize a multidimensional data which is abstract in a more interpretable and displayable format such as on a flat surface or projection in 3D. Finally, the Output of Process is achieved [420] as the output of the transformed distance and if one-dimensional through projection of multidimensional data on a single dimension using a dimensionality reduction method such as determining principal components using principal component analysis and then retaining only the first principal component, then with a numeric risk stratification score, a patient recovery score, if with 2 or more dimensions a color-coded representation of scores, if a sequence of 2 or higher dimensional numbers then a pattern representation of scores and pattern and color-coded representations of scores (wherein the scores are the numerical outputs from [410]) which are the transformed versions of the clustered data through a process such as dimensionality expansion. As a fourth method, alternatively, once subjected to dimensionality reduction, the reduced dimensional data can be projected on to a manifold with dimensionality greater than 1. The inference from performing such transformations of the underlying patient assessment is enhanced from a visualization perspective and can reveal more information depending on how the visualizations are presented. The preferred score is a single dimensional numeric, which is preferred due to the simplicity and ease of interpretation by the clinicians. However, multi-dimensional representations of the score through colors, images, or videos can present more information than a single number and less likely to reduce or lose information by way of reduction to a single numeric value such as risk score. Once projected, the data may be encoded following a scheme that converts the transformed numeric values into representative colors. For example, with the numeric score being provided as the first 3 principal components derived after applying principal component analysis on the multidimensional score, the values of the score can be normalized in all dimensions to range from 0 to 1 using a min-mas scaling normalization method, then the first, second, and third dimensions can be assigned to represent the primary colors red, blue, and green, respectively. This is a means of obtaining a color that can be visualized in lieu of a numerical score into a visual representation such as colors, time-varying colors, patterns, or a combination of colors and patterns that are indicative of patient status. For example, if the transformed data is in 4 dimensions with the 4^{th} dimension as time, and each of the other 3 dimensions represent the primary colors, red, green, and blue, then a video can be obtained.

In Method 5, after the input data [100] is obtained, the input data [100] is processed to detect anomalies [510] by subjecting the data to a transformation and then using anomaly detection algorithms such as isolation forest, local outlier factor, robust covariance, and One-Class support vector machines. Preferably, isolation forests are used for anomaly detection by training Isolation forests detects anomalies using isolation which is defined as how far a data point is to the rest of the data. Isolation forest explicitly isolates anomalies using binary trees, demonstrating a new possibility of a faster anomaly detector that directly targets anomalies without profiling all the normal instances. The Output of Process [520] is then obtained with binary detection of anomaly presence and burden or prevalence of anomalies per epoch. Burden is obtained as the number of anomalies in a given duration of measurement of input data, lower burden translates to fewer anomalies per chose epoch. Although in certain embodiments of the fifth method, the input data used for anomaly detection may be subjected to dimensionality reduction, in the preferred embodiment, dimensionality reduction is not included in the process. The input data can be subjected to anomaly detection techniques for time-series data such as predictive modeling approaches including, but not limited to, Auto Regressive Integrated Moving Average (ARIMA), Seasonal Autoregressive Integrated Moving Average (SARIMA), Generalized Autoregressive Conditional Heteroskedasticity (GARCH), or Vector Auto-Regression (VAR). All these methods are used to predict future values of input data using past and current values of the input data. Deviations of the actual data from the predicted values can be treated as anomalies. The anomalies detected may be presented as a number, or a burden metric which counts the trends in the rate of detection of anomalies Additionally, these metrics could be presented as patterns with or without color coding, and images or videos to aid in interpretation.

In Method 6, after the input data [100] is obtained, it is processed for anomaly detection using anomaly detection algorithms such as isolation forest, local outlier factor, robust covariance, and One-Class support vector machines. Preferably, isolation forests are used for anomaly detection. The anomaly detected data is then compared with additional data obtained from an historic library of recovery patterns or templates for the procedure and the associated demographics [150]. The historic library assumes that the selected input data across all methods is available for consumption for the implementation of this method except from patients who were observed prior to these methods. It is now available as a reference for the described methods. The input data, in terms of available data sources, is at least a subset of the input data used for all methods described, if not a superset (a larger more encompassing and exhaustive set) of all input data that can be obtained. The incremental improvement in terms of a pattern within the historical data set and its accuracy of representation of a specific patient population defined by their demographic, their medical history, and type of procedure, is guaranteed in a statistical sense as the number of patients increases, the sample population i.e., historical data set, is more representative of the actual population provided that the sampling allows true representation of all types of patients to determine the distance measure from known patterns [610] Distance measures are established methods to compare multi-dimensional data to quantify the distance between them to ascertain whether they are similar or different. This may be accomplished using a threshold for the distance computed. The mathematical functions to compute distance may be any one or combination of Euclidean, Manhattan, Mahalanobis, Minkowski, Hamming, and cosine distance. Preferably, Minkowski is used in the exemplary method. The patterns of occurrence of anomalies are then transformed using a mathematical model into a quantitative one or higher dimensional metric[620]. As the patterns of occurrence of anomalies are a series in time showing when and how frequently anomalies were detected, the time-series data can be transformed using a model to represent the data such as Autoregressive (AR), Autoregressive moving average (ARMA), Auto Regressive Integrated Moving Average (ARIMA), Seasonal Autoregressive Integrated Moving Average (SARIMA), Generalized Autoregressive Conditional Heteroskedasticity (GARCH), or Vector Auto-Regression (VAR) and the parameters of such models with a chosen order will be a numerical array or a sequence of numbers. This sequence of numbers can be one- or multi-dimensional. Interpolation and serialization or encoding methodology may be used to convert the single-dimensional time series data into multidimensional data. When an image is specifically 2-dimensional data, higher dimensional data is higher in dimensionality compared to the input data, so it can be any number of dimensions as an upper limit. The dimensionality is practically limited by the computational hardware and the available memory. Once transformed, the higher dimensional data may be used to train convolution neural networks or one of a family of recurrent neural networks inclusive of but not limited to long short-term memory (LSTM) networks or bilateral-LSTM networks. The training of any neural network follows the same steps regardless of the type of neural network. Iteratively, the network is presented with the chosen input data, and computations as specified by the architecture of the neural network, the produced output which in this case is an estimate of assessment of a post-surgical patient is compared against a simultaneously measured value to compute an error which is then used to update the weights or parameters of a neural network during training. The ultimate objective is to iteratively improve the accuracy of values produced by the network by incrementally evolving the parameters within the neural architecture that is defined prior to initiation of training Recurrent neural networks are a type of neural networks where the output of one layer is recurrently presented as an input to the layer. These networks are specifically suitable for time series data inputs and predictions that need to be made using time series data as inputs. There are several nuanced variations to the basic architecture of these networks that may result in variants, but the basic idea of recurrence remains the same—present the output or part of the output of a layer as a part of the input to the layer so that there is memory in the network, or it remembers what value it saw before and modifies its predictions accordingly.

Finally, the Output of Process which is the list of scores is achieved [630] by applying a transformation such as dimensionality expansion to increase the anomalies detected over time which is a single dimensional measure into multiple dimensions, with e.g., a numeric risk stratification score, a patient recovery score, a color-coded representation of scores, a pattern representation of scores and pattern, color-coded representations of scores and combinations thereof. At least 3 dimensions are needed for colors and at least 4 dimensions for color varying over time representation. Therefore, when the mathematical model applied at the end provides higher dimensional results or supports such an output, these additional further representations of the output recovery score are possible. Distance measures from known patterns is a quantity that can represent distance between several of the selected and conditioned inputs such that for each input data selected [100], there can be a distance measure numeric value, so there can be as many values as there are input data. Therefore, when a transformation is applied as in [620] to this list of distance measures, a multidimensional result can be obtained, i.e., one or higher dimensional metric. For example, if the input selected in [100] is a series of 3 data such as number of times a patient awakened between 12 am and 4 am, heart rate when awakened, respiration rate when awakened, for a given surgical procedure performed on a patient with a specific age and gender, then the distance of these data from a historical average of the same data for the same surgery conducted on a patient with the same age and gender is computed in [610] this distance measure combined with an assessment of anomalies detected in the input data consisting of these 3 data results in 6 numbers, 3 for the distances and 3 for the number of anomalies for each data. Thus, we have a 6-dimensional score. This 6-dimensional score can then be transformed to a 3-dimensional score by applying a principal component analysis-based decomposition to result in the first 3 principal components. These 3 numbers can now be used to represent a color with each number representing the strength from a scale of 0-1 for each of the primary colors red, green, and blue.

In Method 6, the output of anomaly detection is compared to historically known patterns of occurrence of anomalies among patients who have undergone similar procedures and belong to the same demographic groups. The output could be presented as a number counting the number of detected anomalies, a burden metric counting the trends in the rate of detection of anomalies, or transformed into a numeric score for risk stratification, recovery index, or visual representations of patterns, color-coded images, or videos. The preferred embodiment is a single numeric risk score or a patient recovery score.

Supervised methods that result in continuous valued outputs such as a numeric score indicative of patient status, recovery, or trajectory of recovery, may include regression techniques such as Tree-based methods, support vector machines, logistic and linear regression with variable coefficients, Gaussian process models, and neural networks. Tree-based methods may include but are not limited to boosted trees, random forests, gradient boosting trees, extreme gradient boosting, AdaBoost, bagged trees, and an ensemble of tree-based models, which is a combination of any or multiple tree-based models. Support vector machines may include but are not limited to linear, cubic, quadratic, coarse, fine, or medium gaussian. Gaussian process regression models may include but are not limited to rational quadratic, squared exponential, exponential, Matern 5/2 kernels. Neural networks may include but are not limited to multilayer perceptrons, generalized regression neural networks, radial basis function networks, recurrent neural networks, long-short term memory networks, and convolution neural networks. All of these supervised machine learning techniques can be applied to the selected, conditioned, and prepared input data to obtain the desired numerical output. Long-short term memory networks and convolution neural networks are preferred among these methods.

Supervised methods that result in classification outputs that could be indicative of different levels of recovery or trajectories toward recovery as opposed to continuous valued assessments. These methods may include but are not limited to logistic regression, support vector machines classifiers, tree-based classifier methods, ensemble methods, naïve Bayes classifiers, family of neural networks such as multilayer perceptrons, autoencoders, recurrent neural networks, long short-term memory networks, convolution neural networks, with the output layer appropriate for a classification output such as but not limited to softmax, taylor softmax, soft-margin softmax, and SM-Taylor softmax. All methods can produce as output reflecting the presence of anomalies Long short-term memory networks with SM-Taylor softmax output layers are preferred.

Labels associated with the data inclusive of outcomes of the surgical intervention, such as patient reported outcomes in the form of surveys, physical, or psychological assessments, quantitative, or qualitative biofluid tests or biopsy images may be used as observed outputs or targets to train a machine learning model inclusive of neural networks using a supervised learning approach. The machine learning model inputs could be any one or combination of several inputs described in the methods for input selection. The outputs may include continuous valued regression outputs, or single- or multi-class classification outputs. In one embodiment, continuous valued regression outputs could be indicative of predicted time to occurrence of the predicted outcome, or likely or probable observation of the label outcome that was observed in the training data set. As such, these outputs could also be transformed into a fixed range of values such as from 0 to 1 or 0 to 100 to indicate a score that is reflective of risk, recovery status, or trajectory of recovery of the patient towards stability or complication. In another embodiment, the class outputs may be one of a set of classes that are indicative of a specific pattern of patient status evolution, given the patient's demographic, history, and time and type of procedure. There is a plurality of approaches to supervised learning to translate transformed inputs into metrics.

FIG. 7 shows a plurality of further methods (Methods 7-13) to formulate model architecture that predict patient status, or trajectory towards recovery after surgical intervention using supervised machine learning methods or combinations of supervised and unsupervised machine learning methods. Method 13 is preferred as anomalous data is emphasized in the steps followed for feature engineering and anomalies are important indicators of any aberration from a normal recovery trajectory controlled by the body's natural mechanisms of maintaining or restoring homeostasis. The detection of anomalies in physiology is important to determine if there are any problems with the patient's recovery. As with Method 6, all the methods are means to affect the same type of transformation from input data to a patient status assessment. These methods result in a continuous valued patient status assessment. The preferred method is long-short term memory networks and convolution neural networks.

In Method 7, after the input data [100] is obtained, a Time Series Regression Neural Networks [710] is performed. The input data for [710] is only data that can be represented as a time series and does not include other data types such as categorical or binary. The data is then submitted to a Model Training and Selection [720] where it is compared to Observed Output Data (one or more) [730] having patient reported outcomes, physical assessment scores and scans or test results indicative of patient status relative to pre-operative status or baseline. The result of this comparison is the Output of Process (one or more) [750] which results in a patient status or recovery trajectory prediction model and anomalous recovery or complication pattern detection model. In Method 7, the input data may be applied to models after conditioning which may involve filtering in time, frequency, wavelet, or other domains using a reversible transformation. The inputs with or without conditioning may be retained in time-series format to be supplied to a class of neural networks or deep learning architectures that have output layers that support continuous-valued regression outputs, or single- or multi-class classification outputs. All the methods above are means to affect the same type of transformation from input data to a patient status assessment. These methods result in methods that may provide a categorical output that is one of a finite number of potential outputs. For example, recovered (output of method=1) or not recovered (output of method=0). The choice of which to use is determined by applying all the methods to the input data and observing which methods provide the most accurate result. The preferred method is long-short term memory networks with SM-Taylor softmax output layers.

In Method 8, after the input data [100] is obtained, a one-dimensional time series to 2 or higher dimensional encoding or sequential mapping leading to time series data [810], for example, input data in the form of images which is a vertical stack of rows of numbers can be sequentially arranged as in every row concatenated to form a long sequence that is fully representative of the image commonly referred to as unraveling a multi-dimensional array, leading to applicability of a time series regression neural networks [710] because a non-sequential data had been effectively converted into a sequence. [810] is performed in case of input data including certain data that are not time series data, after which a Time Series Regression Neural Networks [710] is performed which involves the training of [710] so that the training results in a neural network that is able to take as input time series data by [710] or as arranged by [810]. The data then follows the course of Method 7 with submission to a Model Training and Selection [720] and comparison to Observed Output Data (one or more) [730] having patient reported outcomes, physical assessment scores and scans or test results indicative of patient status relative to pre-operative status or baseline, with a result of the comparison being the Output of Process (one or more) [750] which results in a patient status or recovery trajectory prediction model and anomalous recovery or complication pattern detection model.

In Method 9, features present within the input data are included that are computed or extracted as predefined waveform patterns or time-varying quantities that can be trended, such as but not limited to, distribution of amplitude and power in different frequency ranges of any of the input time-series data. These time-frequency analysis and computation methods may involve any transformation methods such as Fourier, wavelet, short-time Fourier, cepstral analysis, empirical mode decomposition, or wavelet decomposition. Time synchronous features obtained through the fusion of different types of input signals such as but not limited to ECG and heart sound to obtain features such as the R peak to S1, S2, S3, and S4 times, if present, which are reflective of pre-ejection and ejection times from the left ventricle of the heart during the isovolumic contraction period leading up to systole and diastole thereafter. These timing features are differently important for the prediction of cardiac sufficiency following a surgical procedure, and their respective importance may vary based on confounders such as arterial stiffness or pharmacological effects of vasoactive drug therapy.

In Method 9, after the input data [100] is obtained, the waveform pattern characteristics in time and transform domains are extracted [910]. The extracted features [920] are a set of all features that were computed or extracted from the input data where there is simple aggregation of all features matched by the timing of the data that was originally used to extract the features. It simply combines all features into a time synchronous set of data. Then, a feature selection is conducted [930], after which strongly correlated features [940] are determined and then compared to Observed Output Data (one or more) [730] having patient reported outcomes, physical assessment scores and scans or test results indicative of patient status relative to pre-operative status or baseline in a model training and selection [720], with a result of the comparison being the Output of Process (one or more) [750] which results in a patient status or recovery trajectory prediction model and anomalous recovery or complication pattern detection model. In Method 9, all input data may be subjected to unsupervised learning methods to form clusters such as those based on neural networks such as autoencoders, self-organizing maps or adaptive resonance theory-based neural networks, or machine learning techniques such as k-means clustering, Gaussian mixture models, Naïve Bayes, density-based or model-based techniques. Once the clusters are defined in the input feature space or domain, these clusters may in turn, be used for regression-based patient status or recovery trajectory prediction models as features. Attributes of the clusters such as cluster membership or number of data points classified into a particular cluster or distribution across clusters is an example of how clustering outputs may be used as features.

In Method 10, after the input data [100] is obtained, an unsupervised clustering method leading to clustering of data in different levels of granularity of time is performed to obtain extracted features [920] and then a feature selection is conducted [930], after which strongly correlated features [940] are determined and then compared to Observed Output Data (one or more) [730] having patient reported outcomes, physical assessment scores and scans or test results indicative of patient status relative to pre-operative status or baseline in a model training and selection [720], with a result of the comparison being the Output of Process (one or more) [750] which results in a patient status or recovery trajectory prediction model and anomalous recovery or complication pattern detection model. In Method 10, a subset or all layers of a trained neural network may be used as a feature extractor. An example embodiment of this approach may involve the transformation of the input layer of a pre-trained network to accept the input from this device and a subset of all the remaining layers of such a pre-trained neural network or all layers, but the output layer may be retained in the model. The output of the penultimate layer of this neural network may be used as a transformation to extract features that may be the input of a subsequent regression model to predict the patient's status, recovery or trajectory of recovery. In this embodiment, the pre-trained neural network implements a transfer function for the extraction of features. Examples of such neural networks are convolution neural networks, recurrent neural networks, and encoder networks.

In Method 11, after the input data [100] is obtained, a one dimensional time series to 2 or higher dimensional encoding or sequential mapping leading to time series regression neural networks [810] is performed, after which the resulting data is applied to a subset of pretrained neural network layers on a different dataset and the output of the penultimate or earlier layer is used as the extracted features [1110] The extracted features [920] are a set of features expected to be relevant as inputs for the models input to predict recovery or anomalous recovery. [920] conducts a time synchronous aggregation of all features that were extracted and then a feature selection is conducted [930], after which strongly correlated features [940] are determined and then compared to Observed Output Data (one or more) [730] having patient reported outcomes, physical assessment scores and scans or test results indicative of patient status relative to pre-operative status or baseline in a model training and selection [720], with a result of the comparison being the Output of Process (one or more) [750] which results in a patient status or recovery trajectory prediction model and anomalous recovery or complication pattern detection model. In Method 11, the input data may be transformed into an image or higher dimensional space using reversible transformations that do not result in any loss of information. Interpolation and serialization or encoding methodology may be used to convert the single-dimensional time series data into multidimensional data. Once transformed, the higher dimensional neural networks may be used to train convolution neural networks or one of a family of recurrent neural networks inclusive of, but not limited to, long short-term memory (LSTM) networks or bilateral-LSTM networks.

In Method 12, after the input data [100] is obtained, the input data is applied to the discriminator of a trained generative network [1210] to obtain extracted features [920] and then a feature selection is conducted [930], after which strongly correlated features [940] are determined and then compared to Observed Output Data (one or more) [730] having patient reported outcomes, physical assessment scores and scans or test results indicative of patient status relative to pre-operative status or baseline in a model training and selection [720], with a result of the comparison being the Output of Process (one or more) [750] which results in a patient status or recovery trajectory prediction model and anomalous recovery or complication pattern detection model. In Method 12, a generative neural network that consists of structures involving a generator component and a discriminator component is trained, such as a Generative Adversarial Network (GAN). The input data applied to the discriminator generates a set of features that can be used to train another neural network or machine learning model to predict patient status, recovery status, or trajectory towards recovery.

In Method 13, after the input data [100] is obtained, it is compared to Historic Library of recovery patterns or templates for procedure and the associated demographics [1320]. The Historic Library assumes that the selected input data across all methods is available for consumption by the processes described herein, inclusive of all methods, except from patients who were observed prior to these methods. Once this selected input is obtained, the Historic Library is then available as a reference for the described methods. The input data, in terms of available data sources, is at least a subset of the input data used for all methods described, if not a superset (a larger more encompassing and exhaustive set) of all input data that can be obtained. The incremental improvement in terms of a pattern within the historical data set and its accuracy of representation of a specific patient population defined by their demographic, their medical history, and type of procedure, is guaranteed in a statistical sense as the number of patients increases, the sample population i.e., historical data set, is more representative of the actual population. provided that the sampling allows true representation of all types of patients.) to determine the distance measure from known patterns [1310] to in turn determine strongly correlated features [940]. A distance measure is a mathematical function that determines the distance between points in a multidimensional space i.e., if the pattern is 3 dimensional and the data is 3-dimensional, distance measured as Euclidean distance is simply the length of a line segment joining the two points in 3-dimensional space. The strongly correlated features as determined by feature selection algorithms such as minimum redundancy maximal relevance (MRMR), F-statistic or f-test, neighborhood component analysis, backward elimination, forward selection, random forests impurity-based importance, permutation feature importance, factor analysis, and relief algorithm for regression. It is possible to apply all of these different algorithms and then choose the best performing. In one preferred embodiment of the present invention, random forests impurity-based importance and MRMR are used to select features, with a preference for MRMR, are combined with the Observed Output Data (one or more) [730] having patient reported outcomes, physical assessment scores and scans or test results indicative of patient status relative to pre-operative status or baseline in a model training and selection [720], with a result of the comparison being the Output of Process (one or more) [750] which results in a patient status or recovery trajectory prediction model and anomalous recovery or complication pattern detection model. In Method 13, the patterns of change of the patient data from one epoch to the next, could be associated with metadata such as the patient's demographics, and type of procedure to form a library of templates that characterize the typical expected trajectory of change towards recovery. (It is noted that “epoch” here refers to a duration of time over which the patient status, recovery score, or risk stratification is computed.) The distance from known patterns from patients is essentially a mathematical calculation of distance to determine similarity or difference between two data. The mathematical functions to compute distance may be any one or combination of Euclidean, Manhattan, Mahalanobis, Minkowski, Hamming, and cosine distance, with similar demographics undergoing similar procedures can be used as strongly correlated features for supervised learning. New patient data could be matched against these templates to perform anomaly detection and raise alerts or alarms to emphasize patients who require additional clinician attention. In this embodiment, the patient status or recovery assessment model could be used as a segmentation and triaging tool by clinicians across a clinical practice or healthcare provider.

Method 14, a preferred embodiment which appears in FIG. 5, uses steps from Method 1 and Method 9. In Method 14, the input data is obtained [100], and the waveform pattern characteristics in time and transform domains are extracted [1410]. The extracted features [1420] are a set of all features that were computed or extracted from the input data. The extracted features [1420] are then subjected to clustering methods [110], after which the output of the clustering methods (the cluster membership) is transformed by mathematical model [120], an example of such as model is—

$$Quantified {Change}_{i} = \vert \frac{{clustercount}_{i}^{n} - {clustercout}_{i}^{n - 1}}{{clustercount}_{i}^{n} + {clustercout}_{i}^{n - 1}} \vert$$

where, i is the cluster number, and n and n−1, refer to the member counts in the ith cluster for the current and previous epoch (An epoch is the duration of time over which the input data spans), respectively, into a one-dimensional metric [120]. Finally, the output of the process, which is the result of the mathematical formula or model [120], is obtained [130] (following the example the obtained output is the quantified change per epoch) resulting in a numeric risk stratification score which is indicative of the likelihood of a complication leading to recovery or worsening of the patient's health following a surgery and a patient recovery score, complementary to the risk stratification score indicates the likelihood of recovery to baseline conditions after surgery. The scores do not provide an assessment of health. They provide an assessment of change after a surgery, e.g., an assessment of change in the physiological, behavioral, and cognitive status of patients. The preferred embodiment uses-rule based algorithms to calculate all the features listed in Table 1.

Similarly, a personalized status or recovery trajectory model, using input data from the patient and also using historical data just from that patient, may further be developed through one of the following approaches:
Transfer learning wherein a pre-trained model on a population may be trained further with data from an individual to generate a prediction model that is unique for that individual, with preferred embodiments having large populations comprising over 50 subjects.
Training target data may be obtained through a periodic assessment technique inclusive of patient reported outcomes and physiological or psychological measures of the patient's status with periodicity of assessment ranging from a few minutes to a few days. These measurements may be used as a personalized training set for an individual and an exploration of all aforementioned feature extraction and model training methods may be applied to create a customized patient status or recovery, or recovery trajectory prediction model.
The time series observations or transformed inputs trended over time for a patient may be subjected to anomaly detection techniques such as IQR, K-means clustering, or Isolation Forest.

An exemplary implementation of this methodology is found in Method 14 and the exemplary implementation as described in FIG. 1. However, a more generalized approach for each of the three steps is considered a natural extension of this example for those skilled in the art. Further, this may be applied to a wide array of conditions depending on potential for surgical complications ranging from minor risk elective surgeries to major risk cardiovascular or cerebrovascular surgical interventions.

### Step 1—Input Selection

The most direct and near deterministic inputs that may be used to assess patient status are physiological vital signs or waveforms, such as but not limited to ECG, heart sounds, thoracic impedance, posture, and activity. These inputs may be measured at any level of granularity in time, synchronously or asynchronously using a single device or multiple devices. An additional step to input selection is the selection of the stage of perioperative care when vital signs monitoring should commence. In this embodiment, the monitoring may start 24 hours before the index surgical intervention event, which may be the start of procedure or start of the preoperative procedures. Monitoring may extend from this event through the intraoperative stage and continue to the post-operative stage.

### Step 2—Transformation of Inputs

The raw input data may be transformed into features such as but not limited to heart rate, heart rate variability, respiration effort and rate, systolic and diastolic blood pressure, blood oxygen saturation, posture, angle of inclination of the upper and lower body, and activity. These features maybe aggregated into different levels of granularity in time. In this exemplary embodiment, features are aggregated to minute level granularity. Statistical measures of the variability of these aggregated vital signs in terms of mean, median, standard deviation could be further transformations that have physiological correlates to the process of recovery. Table 1 presents an exemplary transformation of time-synchronously acquired 2 channels of ECG waveforms, 2 channels of thoracic impedance, 3-axis accelerometer, and 1 channel of heart sounds into a plurality of dimensions of features. The selection of transformation for the inputs to convert them into features that are relevant to surgical recovery consist of methods such as rule-based determination of certain features from the input data.

TABLE 1
List of exemplary transformations of measured input data to extraction and derivation of
features and dimensions.

| Exemplary | | | |
|---|---|---|---|
| metric | | | |
| number | Variable Name | Units | Description |
| 1. | Measurement Time | Datetime | The time in this variable provides the date and time of |
| | | | day at which the measurement was made based on the |
| | | | data collected by the SimpleSENSE device. |
| 2. | MeanHeartRate | Beats per | Average heart rate computed over the measurement |
| | | minute | minute. Both ECG and Heart sounds are used to |
| | | | compute the mean heart rate. |
| 3. | SDNN | Millisecon | The standard deviation of the inter-beat interval (IBIs) |
| | | ds | of normal sinus beats (SDNN) is measured in ms. |
| | | | “Normal” means that abnormal beats, like ectopic beats |
| | | | (heartbeats that originate outside the atrium’s sinoatrial |
| | | | node), have been removed. |
| 4. | MedianHeartRate | Beats per | Median heart rate computed over the measurement |
| | | minute | minute. Both ECG and Heart sounds are used to |
| | | | compute the mean heart rate. |
| 5. | S1amplitudeRMSMean | Volts | Mean of the root mean square (RMS) amplitude over a |
| | | | 125-millisecond window centered at the time of |
| | | | occurrence of the S1 sound of the heart sound. A single |
| | | | value, the mean of the RMS, is computed from a minute |
| | | | of heart sound signal. |
| 6. | S1amplitudeRMS Std | Volts | Standard deviation (STD) of the root mean square |
| | | | amplitude over a 125-millisecond window centered at |
| | | | the time of occurrence of the S1 sound of the heart |
| | | | sound. A single value, the STD of the RMS, is |
| | | | computed from a minute of heart sound signal. |
| 7. | S2amplitudeRMSMean | Volts | Mean of the root mean square (RMS) amplitude over a |
| | | | 125-millisecond window centered at the time of |
| | | | occurrence of the S2 sound of the heart sound. A single |
| | | | value, the mean of the RMS, is computed from a minute |
| | | | of heart sound signal. |
| 8. | S2amplitudeRMSStd | Volts | Standard deviation (STD) of the root mean square |
| | | | amplitude over a 125-millisecond window centered at |
| | | | the time of occurrence of the S2 sound of the heart |
| | | | sound. A single value, the STD of the RMS, is |
| | | | computed from a minute of heart sound signal. |
| 9. | S3amplitudeRMSMean | Volts | Mean of the root mean square (RMS) amplitude over a |
| | | | 125-millisecond window centered at a time 72 |
| | | | milliseconds after the detected S2 sound peak. A single |
| | | | value, the mean of the RMS, is computed from a minute |
| | | | of heart sound signal. |
| 10. | S3amplitudeRMSStd | Volts | STD of the root mean square (RMS) amplitude over a |
| | | | 125-millisecond window centered at a time 72 |
| | | | milliseconds after the detected S2 sound peak. A single |
| | | | value, the STD of the RMS values, is computed from a |
| | | | minute of heart sound signal. |
| 11. | S1amplitudePEAKMean | Volts | Peak amplitude over a 125-millisecond window |
| | | | centered at the time of occurrence of the S1 sound of the |
| | | | heart sound. A single value, the mean of the peak |
| | | | values, is computed from a minute of heart sound signal. |
| 12. | S1amplitudePEAKStd | Volts | STD of the Peak amplitude over a 125-millisecond |
| | | | window centered at the time of occurrence of the S1 |
| | | | sound of the heart sound. A single value, the mean of |
| | | | the peak values, is computed from a minute of heart |
| | | | sound signal. |
| 13. | S2amplitudePEAKMean | Volts | Peak amplitude over a 125-millisecond window |
| | | | centered at the time of occurrence of the S2 sound of the |
| | | | heart sound. A single value, the mean of the peak |
| | | | values, is computed from a minute of heart sound signal. |
| 14. | S2amplitudePEAKStd | Volts | STD of the Peak amplitude over a 125-millisecond |
| | | | window centered at the time of occurrence of the S2 |
| | | | sound of the heart sound. A single value, the mean of |
| | | | the peak values, is computed from a minute of heart |
| | | | sound signal. |
| 15. | S3amplitudePEAKMean | Volts | Peak amplitude over a 125-millisecond window |
| | | | centered at time 72 milliseconds after the detected S2 |
| | | | sound peak. A single value, the mean of the peak |
| | | | amplitudes, is computed from a minute of heart sound |
| | | | signal. |
| 16. | S3amplitudePEAKStd | Volts | STD of the peak amplitude over a 125-millisecond |
| | | | window centered at a time 72 milliseconds after the |
| | | | detected S2 sound peak. A single value, the STD of the |
| | | | peak values, is computed from a minute of heart sound |
| | | | signal. |
| 17. | respirationRateMean1 | Breaths per | Mean respiration rate computed over a minute of |
| | | minute | thoracic impedance data from channel 1 from the three |
| | | | methods - sine fit, burg power spectrum and peak count. |
| 18. | respirationRateMedian1 | Breaths per | Median respiration rate computed over a minute of |
| | | minute | thoracic impedance data from channel 1 from the three |
| | | | methods - sine fit, burg power spectrum and peak count. |
| 19. | respirationRateStd1 | Breaths per | STD of respiration rate computed over a minute of |
| | | minute | thoracic impedance data from channel 1 from the three |
| | | | methods - sine fit, burg power spectrum and peak count. |
| 20. | respirationRateMean_burg1 | Breaths per | Mean respiration rate computed over a minute of |
| | | minute | thoracic impedance data from channel 1. A power |
| | | | spectral estimate is computed using the burg auto |
| | | | regressive algorithm. The frequency associated with the |
| | | | most dominant sinusoid is treated as the respiratory |
| | | | frequency. This frequency is converter to a per minute |
| | | | value and reported. The reported value is the mean of |
| | | | the respiration rates over one minute of data. |
| | | | Respiration rates using the power spectrum from the |
| | | | burg algorithm over 30 second segments with a 50% |
| | | | overlap. |
| 21. | respirationRateMedian_burg1 | Breaths per | Median respiration rate computed over a minute of |
| | | minute | thoracic impedance data from channel 1. A power |
| | | | spectral estimate is computed using the burg auto |
| | | | regressive algorithm. The frequency associated with the |
| | | | most dominant sinusoid is treated as the respiratory |
| | | | frequency. This frequency is converter to a per minute |
| | | | value and reported. The reported value is the median of |
| | | | the respiration rates over one minute of data. |
| | | | Respiration rates using the power spectrum from the |
| | | | burg algorithm are computed over 30 second segments |
| | | | of data with a 50% overlap. |
| 22. | respirationRateStd_burg1 | Breaths per | STD of respiration rates computed over a minute of |
| | | minute | thoracic impedance data from channel 1. A power |
| | | | spectral estimate is computed using the burg auto |
| | | | regressive algorithm. The frequency associated with the |
| | | | most dominant sinusoid is treated as the respiratory |
| | | | frequency. This frequency is converter to a per minute |
| | | | value and reported. The reported value is the STD of the |
| | | | respiration rates over one minute of data. Respiration |
| | | | rates using the power spectrum from the burg algorithm |
| | | | are computed over 30 second segments of data with a 50% |
| | | | overlap. |
| 23. | respirationRateMean_ | Breaths per | Mean respiration rate computed over a minute of |
| | peakcount1 | minute | thoracic impedance data from channel 1. Respiration |
| | | | rates are calculated by counting the number of peaks in |
| | | | the raw respiration waveform. Respiration rates using |
| | | | the peak count algorithm are computed for over 30 |
| | | | second segments with 50% overlap. The reported value |
| | | | is the mean of the respiration rates over one minute of |
| | | | data. |
| 24. | respirationRateMedian_ | Breaths per | Median respiration rate computed over a minute of |
| | peakcount1 | minute | thoracic impedance data from channel 1. Respiration |
| | | | rates are calculated by counting the number of peaks in |
| | | | the raw respiration waveform. Respiration rates using |
| | | | the peak count algorithm are computed for over 30 |
| | | | second segments with 50% overlap. The reported value |
| | | | is the median of the respiration rates over one minute of |
| | | | data. |
| 25. | respirationRateStd_ | Breaths per | STD of respiration rate computed over a minute of |
| | peakcount1 | minute | thoracic impedance data from channel 1. Respiration |
| | | | rates are calculated by counting the number of peaks in |
| | | | the raw respiration waveform. Respiration rates using |
| | | | the peak count algorithm are computed for over 30 |
| | | | second segments with 50% overlap. The reported value |
| | | | is the STD of the respiration rates over one minute of |
| | | | data. |
| 26. | respirationRateMean_ | Breaths per | Mean of respiration rates computed over a minute of |
| | sinefit1 | minute | thoracic impedance data from channel 1. A non-linear |
| | | | optimization method is used to find the frequency and |
| | | | amplitude of the best fit sinusoid over the respiration |
| | | | signal derived from thoracic impedance. The frequency |
| | | | of the best fit sinusoid is then converted to a per minute |
| | | | value and reported as respiration rate. Respiration rates |
| | | | using the sine fit algorithm are computed for over 30 |
| | | | second segments with 50% overlap. The reported value |
| | | | is the mean of the respiration rates over one minute of |
| | | | data. |
| 27. | respirationRateMedian_ | Breaths per | Median of respiration rates computed over a minute of |
| | sinefit1 | minute | thoracic impedance data from channel 1. A non-linear |
| | | | optimization method is used to find the frequency and |
| | | | amplitude of the best fit sinusoid over the respiration |
| | | | signal derived from thoracic impedance. The frequency |
| | | | of the best fit sinusoid is then converted to a per minute |
| | | | value and reported as respiration rate. Respiration rates |
| | | | using the sine fit algorithm are computed for over 30 |
| | | | second segments with 50% overlap. The reported value |
| | | | is the median of the respiration rates over one minute of |
| | | | data. |
| 28. | respirationRateStd_sinefit1 | Breaths per | STD of respiration rates computed over a minute of |
| | | minute | thoracic impedance data from channel 1. A non-linear |
| | | | optimization method is used to find the frequency and |
| | | | amplitude of the best fit sinusoid over the respiration |
| | | | signal derived from thoracic impedance. The frequency |
| | | | of the best fit sinusoid is then converted to a per minute |
| | | | value and reported as respiration rate. Respiration rates |
| | | | using the sine fit algorithm are computed for over 30 |
| | | | second segments with 50% overlap. The reported value |
| | | | is the STD of the respiration rates over one minute of |
| | | | data. |
| 29. | respirationRateTidalVol- | Ohms | Mean of the ratio of the range of thoracic impedance |
| | umeRatioMeanl | | values measured within a minute of SimpleSENSE data |
| | | | from channel 1 to the corresponding respiration rates. |
| | | | These ranges are computed for over 30 second segments |
| | | | with 50% overlap. |
| 30. | respirationRateTidalVol- | Ohms | Median of the ratio of the range of thoracic impedance |
| | umeRatioMedianl | | values measured within a minute of SimpleSENSE data |
| | | | from channel 1 to the corresponding respiration rates. |
| | | | These ranges are computed for over 30 second segments |
| | | | with 50% overlap. |
| 31. | relativeTidalVolumeMean1 | Ohms | Mean of the range of thoracic impedance values |
| | | | measured within a minute of SimpleSENSE data from |
| | | | channel 1. These ranges are computed for over 30 |
| | | | second segments with 50% overlap. |
| 32. | relativeTidalVolumeMedian1 | Ohms | Median of the range of thoracic impedance values |
| | | | measured within a minute of SimpleSENSE data from |
| | | | channel 1. These ranges are computed for over 30 |
| | | | second segments with 50% overlap. |
| 33. | respirationRateMean2 | Breaths per | Mean respiration rate computed over a minute of |
| | | minute | thoracic impedance data from channel 2 from the three |
| | | | methods - sine fit, burg power spectrum and peak count. |
| 34. | respirationRateMedian2 | Breaths per | Median respiration rate computed over a minute of |
| | | minute | thoracic impedance data from channel 2 from the three |
| | | | methods - sine fit, burg power spectrum and peak count. |
| 35. | respirationRateS td2 | Breaths per | STD of respiration rate computed over a minute of |
| | | minute | thoracic impedance data from channel 2 from the three |
| | | | methods - sine fit, burg power spectrum and peak count. |
| 36. | respirationRateMean_bu | Breaths per | Mean respiration rate computed over a minute of |
| | rg2 | minute | thoracic impedance data from channel 2. A power |
| | | | spectral estimate is computed using the burg auto |
| | | | regressive algorithm. The frequency associated with the |
| | | | most dominant sinusoid is treated as the respiratory |
| | | | frequency. This frequency is converter to a per minute |
| | | | value and reported. The reported value is the mean of |
| | | | the respiration rates over one minute of data. |
| | | | Respiration rates using the power spectrum from the |
| | | | burg algorithm over 30 second segments with a 50% |
| | | | overlap. |
| 37. | respirationRateMedian_burg2 | Breaths per | Median respiration rate computed over a minute of |
| | | minute | thoracic impedance data from channel 2. A power |
| | | | spectral estimate is computed using the burg auto |
| | | | regressive algorithm. The frequency associated with the |
| | | | most dominant sinusoid is treated as the respiratory |
| | | | frequency. This frequency is converter to a per minute |
| | | | value and reported. The reported value is the median of |
| | | | the respiration rates over one minute of data. |
| | | | Respiration rates using the power spectrum from the |
| | | | burg algorithm are computed over 30 second segments |
| | | | of data with a 50% overlap. |
| 38. | respirationRateStd_burg2 | Breaths per | STD of respiration rates computed over a minute of |
| | | minute | thoracic impedance data from channel 2. A power |
| | | | spectral estimate is computed using the burg auto |
| | | | regressive algorithm. The frequency associated with the |
| | | | most dominant sinusoid is treated as the respiratory |
| | | | frequency. This frequency is converter to a per minute |
| | | | value and reported. The reported value is the STD of the |
| | | | respiration rates over one minute of data. Respiration |
| | | | rates using the power spectrum from the burg algorithm |
| | | | are computed over 30 second segments of data with a 50% |
| | | | overlap. |
| 39. | respirationRateMean_ | Breaths per | Mean respiration rate computed over a minute of |
| | peakcount2 | minute | thoracic impedance data from channel 2. Respiration |
| | | | rates are calculated by counting the number of peaks in |
| | | | the raw respiration waveform. Respiration rates using |
| | | | the peak count algorithm are computed for over 30 |
| | | | second segments with 50% overlap. The reported value |
| | | | is the mean of the respiration rates over one minute of |
| | | | data. |
| 40. | respirationRateMedian_ | Breaths per | Median respiration rate computed over a minute of |
| | peakcount2 | minute | thoracic impedance data from channel 2. Respiration |
| | | | rates are calculated by counting the number of peaks in |
| | | | the raw respiration waveform. Respiration rates using |
| | | | the peak count algorithm are computed for over 30 |
| | | | second segments with 50% overlap. The reported value |
| | | | is the median of the respiration rates over one minute of |
| | | | data. |
| 41. | respirationRateStd_peak | Breaths per | STD of respiration rate computed over a minute of |
| | count2 | minute | thoracic impedance data from channel 2. Respiration |
| | | | rates are calculated by counting the number of peaks in |
| | | | the raw respiration waveform. Respiration rates using |
| | | | the peak count algorithm are computed for over 30 |
| | | | second segments with 50% overlap. The reported value |
| | | | is the STD of the respiration rates over one minute of |
| | | | data. |
| 42. | respirationRateMean_sinefit2 | Breaths per | Mean of respiration rates computed over a minute of |
| | | minute | thoracic impedance data from channel 2. A non-linear |
| | | | optimization method is used to find the frequency and |
| | | | amplitude of the best fit sinusoid over the respiration |
| | | | signal derived from thoracic impedance. The frequency |
| | | | of the best fit sinusoid is then converted to a per minute |
| | | | value and reported as respiration rate. Respiration rates |
| | | | using the sine fit algorithm are computed for over 30 |
| | | | second segments with 50% overlap. The reported value |
| | | | is the mean of the respiration rates over one minute of |
| | | | data. |
| 43. | respirationRateMedian_ | Breaths per | Median of respiration rates computed over a minute of |
| | sinefit2 | minute | thoracic impedance data from channel 2. A non-linear |
| | | | optimization method is used to find the frequency and |
| | | | amplitude of the best fit sinusoid over the respiration |
| | | | signal derived from thoracic impedance. The frequency |
| | | | of the best fit sinusoid is then converted to a per minute |
| | | | value and reported as respiration rate. Respiration rates |
| | | | using the sine fit algorithm are computed for over 30 |
| | | | second segments with 50% overlap. The reported value |
| | | | is the median of the respiration rates over one minute of |
| | | | data. |
| 44. | respirationRateS td_sinefit2 | Breaths per | STD of respiration rates computed over a minute of |
| | | minute | thoracic impedance data from channel 2. A non-linear |
| | | | optimization method is used to find the frequency and |
| | | | amplitude of the best fit sinusoid over the respiration |
| | | | signal derived from thoracic impedance. The frequency |
| | | | of the best fit sinusoid is then converted to a per minute |
| | | | value and reported as respiration rate. Respiration rates |
| | | | using the sine fit algorithm are computed for over 30 |
| | | | second segments with 50% overlap. The reported value |
| | | | is the STD of the respiration rates over one minute of |
| | | | data. |
| 45. | respirationRateTidalVol- | Ohms | Mean of the ratio of the range of thoracic impedance |
| | umeRatioMean2 | | values measured within a minute of SimpleSENSE data |
| | | | from channel 2 to the corresponding respiration rates. |
| | | | These ranges are computed for over 30 second segments |
| | | | with 50% overlap. |
| 46. | respirationRateTidalVol- | Ohms | Median of the ratio of the range of thoracic impedance |
| | umeRatioMedian2 | | values measured within a minute of SimpleSENSE data |
| | | | from channel 2 to the corresponding respiration rates. |
| | | | These ranges are computed for over 30 second segments |
| | | | with 50% overlap. |
| 47. | relativeTidalVolumeMean2 | Ohms | Mean of the range of thoracic impedance values |
| | | | measured within a minute of SimpleSENSE data from |
| | | | channel 2. These ranges are computed for over 30 |
| | | | second segments with 50% overlap. |
| 48. | relativeTidalVolumeMedian2 | Ohms | Median of the range of thoracic impedance values |
| | | | measured within a minute of SimpleSENSE data from |
| | | | channel 2. These ranges are computed for over 30 |
| | | | second segments with 50% overlap. |
| 49. | R_S1IntervalMean | Seconds | Mean of the time intervals between the occurrence of |
| | | | the R peak in the ECG and the S1 heart sound peak in |
| | | | the Heart sound signal over a minute of data. |
| 50. | R_S1IntervalMedian | Seconds | Median of the time intervals between the occurrence of |
| | | | the R peak in the ECG and the S1 heart sound peak in |
| | | | the Heart sound signal over a minute of data. |
| 51. | R_S2IntervalMean | Seconds | Mean of the time intervals between the occurrence of |
| | | | the R peak in the ECG and the S2 heart sound peak in |
| | | | the Heart sound signal over a minute of data. |
| 52. | R_S2IntervalMedian | Seconds | Median of the time intervals between the occurrence of |
| | | | the R peak in the ECG and the S2 heart sound peak in |
| | | | the Heart sound signal over a minute of data. |
| 53. | PostureMedian | Categorical/ | Median posture over a minute of data. The strings are |
| | | String | one of the following - ‘Sit/Stand’, ‘Left Lateral’, ‘Right |
| | | | Lateral’, ‘Supine’, or ‘Prone’ |
| 54. | ActivityMean | MilliGs | Mean of the Activity levels measured in milliGs over a |
| | | | minute of data. The Activity is defined as the amount of |
| | | | acceleration measured beyond the acceleration due to |
| | | | gravity measured by the accelerometer. |
| 55. | azimuth | Degrees | The XYZ cartesian coordinate acceleration values are |
| | | | transformed to spherical coordinates. Mean of the |
| | | | azimuthal angle over one minute is reported in this |
| | | | variable. This is associated with orientation within a |
| | | | posture subgroup. For example: - extent of inclination |
| | | | towards a particular posture. Posture medians alone are |
| | | | sensitive to boundary posture states such as inclined |
| | | | postures maybe classified as supine. Azimuthal provides |
| | | | additional information to distinguish these states. |
| 56. | elevation | Degrees | The XYZ cartesian coordinate acceleration values are |
| | | | transformed to spherical coordinates. Mean of the |
| | | | elevation angle over one minute is reported in this |
| | | | variable. This is associated with orientation within a |
| | | | posture subgroup. For example: - extent of inclination |
| | | | towards a particular posture. Posture medians alone are |
| | | | sensitive to boundary posture states such as inclined |
| | | | postures maybe classified as supine. Elevation provides |
| | | | additional information to distinguish these states. |
| 57. | peak_dzdt_l | No units - | Ensemble mean of the ICG waveform from thoracic |
| | | ratio | impedance 1 is computed over one minute. The ratio of |
| | | | the peak of the waveform to the overall root mean |
| | | | square of the ensemble average is reported. |
| 58. | peak_dzdt_2 | No units - | Ensemble mean of the ICG waveform from thoracic |
| | | ratio | impedance 2 is computed over one minute. The ratio of |
| | | | the peak of the waveform to the overall root mean |
| | | | square of the ensemble average is reported. |
| 59. | impedance_l_mean | Ohms | Mean of the thoracic impedance values from channel 1 |
| | | | over one minute. |
| 60. | impedance_2_mean | Ohms | Mean of the thoracic impedance values from channel 2 |
| | | | over one minute. |
| 61. | recording_length | Minutes | Total length of the recording from which the minute |
| | | | data was computed. |
| 62. | SDRR | Milliseconds | The standard deviation of the IBIs for all sinus beats |
| | | | (SDRR). |
| 63. | RMSSD | Milliseconds | The root mean square of successive differences between |
| | | | normal heartbeats (RMSSD) is obtained by first |
| | | | calculating each successive time difference between |
| | | | heartbeats in ms. Then, each of the values is squared and |
| | | | the ed before the square root of the total is obtained. |
| 64. | NN50 | Count | The number of adjacent NN intervals that differ from |
| | | (number) | each other by more than 50 ms (NN50) requires a 2 min |
| | | | epoch. |
| 65. | pNN50 | Percentage | The percentage of adjacent NN intervals that differ |
| | | | from each other by more than 50 ms (pNN50) also |
| | | | requires a 2-min epoch. |
| 66. | SDANN | Milliseconds | The standard deviation of the average normal-to-normal |
| | | | (NN) intervals for each of the 5 min segments during a |
| | | | 24 h recording (SDANN) is measured and reported in |
| | | | ms like the SDNN. This refers to IBIs calculated after |
| | | | artifacting the data. SDANN is not a surrogate for |
| | | | SDNN since it is calculated using 5 min segments |
| | | | instead of an entire 24 h time series. |
| 67. | SDNNI | Milliseconds | The SDNNI is the mean of the standard deviations of all |
| | | | the NN intervals for each 5 min segment of a 24-h HRV |
| | | | recording. Therefore, this measurement only estimates |
| | | | variability due to the factors affecting HRV within a 5- |
| | | | min period. |
| 68. | HRmax_HRmin | Beats Per | The average difference between the highest and lowest |
| | | Minute | HRs during each respiratory cycle (HR Max - HR Min) |
| | | | is especially sensitive to the effects of respiration rate, |
| | | | independent of vagus nerve traffic. At least a 2-min |
| | | | sample is required to calculate HR Max - HR Min. |
| | | | Instead of directly indexing vagal tone, it reflects RSA. |
| | | | Since longer exhalations allow greater acetylcholine |
| | | | metabolism, slower respiration rates can produce higher |
| | | | A amplitudes that are not mediated by changes in vagal |
| | | | firing. |
| 69. | HR V_tri angular_index | Ratio - no | The HTI is a geometric measure based on 24 h |
| | | units | recordings which calculates the integral of the density of |
| | | | the RR interval histogram divided by its height. A 5-min |
| | | | epoch is conventionally used to represent this metric. |
| | | | HTI and RMSSD can joint distinguish between normal |
| | | | heart rhythms and arrhythmias. |
| 70. | TINN | Milliseconds | The TINN is the baseline width of a histogram |
| | | | displaying NN intervals |
| 71. | LF | beat² sec | Total power in the LF frequency band (0.04-0.15 Hz) of |
| | | | the RR interval series over at least 2 minutes of data. |
| 72. | HF | beat² sec | Total power in the HF frequency band (0.15-0.40 Hz) |
| | | | of the RR interval series over at least 2 minutes of data. |
| 73. | LFHFratio | Ratio - No | Ratio of the power in the LF band to the power in the |
| | | units | HF band of the RR interval series. |
| 74. | VLF | beat² sec | Total power in the VLF frequency band (0.0033-0.04 |
| | | | Hz) of the RR interval series over at least 2 minutes of |
| | | | data. |
| 75. | SD1 | Seconds | A non-linear heart rate variability metric - We can |
| | | | analyze a Poincare plot by fitting an ellipse (curve |
| | | | which resembles a squashed circle) to the plotted points. |
| | | | After fitting the ellipse, we can derive three non-linear |
| | | | measurements, S, SD1, and SD2. The area of the ellipse |
| | | | which represents total HRV (S) correlates with |
| | | | baroreflex sensitivity (BRS), LF and HF power, and |
| | | | RMSSD. |
| | | | The standard deviation (hence SD) of the distance of |
| | | | each point from the y = x axis (SD1), specifies the |
| | | | ellipse’s width. SD1 measures short-term HRV in ms |
| | | | and correlates with baroreflex BRS), which is the |
| | | | change in IBI duration per unit change in BP, and HF |
| | | | power. |
| 76. | SD2 | Seconds | The standard deviation of each point from the y = x + |
| | | | average R-R interval (SD2) specifies the ellipse’s |
| | | | length. SD2 measures short- an s and correlates with LF |
| | | | power |
| | | | and BRS. |
| 77. | SD1SD2ratio | Ratio - no | Ratio of SD1 to SD2 |
| | | units | |
| 78. | ApEn | Arbitrary | Approximate entropy measures the regularity and |
| | | units | complexity of a time series. ApEn was designed for |
| | | | brief time series in which some noise may be present |
| | | | and makes no assumptions regarding underlying system |
| | | | dynamics. Applied to HRV data, large ApEn values |
| | | | indicate low predictability of fluctuations in successive |
| | | | RR intervals. Small ApEn that the signal is |
| | | | regular and predictable. |
| 79. | SampEn | Arbitrary | Sample entropy was designed to provide a less biased |
| | | units | and more reliable measure of signal regularity and |
| | | | complexity. SampEn values are interpreted and used |
| | | | like ApEn and may be calculated from a much shorter |
| | | | time series of fewer than 200 values. |
| 80. | DF A_ Alpha1 | Arbitrary | Detrended fluctuation analysis extracts the correlation |
| | | units | between successive RR intervals over different time |
| | | | scales. |
| | | | Alpha 1 slope parameter describes brief fluctuations. |
| 81. | DFA_Alpha2 | Arbitrary | Alpha 2 slope parameter describes long-term |
| | | Units | fluctuations. |
| 82. | CD | Numerical | The CD (D2) estimates the minimum number of |
| | | (maybe | variables required to construct a model of system |
| | | fractional) | dynamics. |
| 83. | CPC_ratio | Ratio - No | A computed term known as cardio-pulmonary coupling |
| | | units | which refers to a quantification of the extent of coupling |
| | | | between respiration and heart rate maintained by |
| | | | autonomous regulation. The basic idea is that the |
| | | | coupling observed at low frequencies and high |
| | | | frequencies will be different for different stages of |
| | | | sleep. Also, the coupling will have less high frequency |
| | | | interaction in case of patients with sleep disorders. It is |
| | | | computed as the product of the coherence and the cross |
| | | | spectral power between the RR intervals and respiration. |
| | | | Ratio is the ratio of LF CPC to HF CPC |
| 84. | CPC_VLF | V² sec | CPC in the VLF frequency band (<0.01 Hz) |
| 85. | CPC_LF | V² sec | CPC in the LF frequency band (0.01-0.1 Hz) |
| 86. | CPC_HF | V² sec | CPC in the HF frequency band (0.1-0.4 Hz) |

The features of Table 2 below are generic physiological assessments that are fundamental measures of physiology, so they are all related to surgical recovery.

TABLE 2
Exemplative Features

| Name of Feature | Source | Description |
|---|---|---|
| Atrial Electrical Activity | ECG | Duration and |
| | | amplitude of P wave of |
| | | ECG waveform |
| Ventricular Electrical | ECG | Duration and |
| Activity | | amplitude of QRS |
| | | wave of ECG |
| | | waveform |
| PR interval or Atrio- | ECG | Time elapsed between |
| ventricular conduction | | P wave and R wave |
| interval | | occurrence in an ECG |
| QRS measures | ECG | Amplitude, duration |
| | | and axis of QRS waves |
| | | of ECG. |
| ST-T wave measures | ECG | ST segment amplitude, |
| | | duration and slope in |
| | | ECG waveform |
| S1, S2, S3, S4 sounds | Heart sound | Prescence, amplitude, |
| | | magnitude, loudness, |
| | | time durations of heart |
| | | sounds |
| Other heart sounds and | Heart sound | Prescence, amplitude, |
| noise | | magnitude, loudness, |
| | | time durations of heart |
| | | sounds and noise |
| Patient Activity Score | Actigraphy | Measure of physical |
| | | activity performed by |
| | | patient like walking, |
| | | climbing stairs or more |
| | | intense exercise |
| Posture | Actigraphy | Measure of the |
| | | absolute posture |
| | | maintained |
| Cardiac Output | ICG | Measure of the volume |
| | | of blood pumped from |
| | | the heart in a minute. It |
| | | is the product of the |
| | | volume of blood |
| | | pumped out be the left |
| | | ventricle of the heart |
| | | and the heart rate. |
| Stroke Volume | ICG | Measure of volume of |
| | | heart pumped from the |
| | | ventricle of the heart. |
| Cardio-vascular Pressures | ICG | Measure of the |
| | | maximum pressure in |
| | | the blood vessels |
| | | following a heart |
| | | muscle contraction that |
| | | causes blood flow and |
| | | pressure between |
| | | consecutive |
| | | contractions. |
| Patient Geographic | Smart devices | Global Positioning |
| Location and Altitude | | Satellite (GPS) |
| | | location of the patient |
| | | from the patient’s |
| | | smart device. |
| Pulmonary Measures | ICG | Pulmonary measures |
| | | include tidal volume - |
| | | volume of air inhaled |
| | | or exhaled during |
| | | normal breathing, and |
| | | rate of breathing, |
| | | whether the patient is |
| | | experiencing shortness |
| | | of breath wherein the |
| | | respiratory rate is high, |
| Name of Feature | Source | Description |
| | | but the volume of air |
| | | displaced from the |
| | | lungs is low. |
| Minute Ventilation | ICG | Measure of the amount |
| | | of air displaced by the |
| | | lungs in a minute. |
| Shortness of Breath | ICG and ECG | Measure of the amount |
| | | of air inhaled or |
| | | exhaled and rate of |
| | | respiration. |
| Exercise Tolerance | ECG and ICG | Measures the changes |
| | | in heart rate and |
| | | respiration while |
| | | performing activities |
| | | like a 6-minute walk |
| Heart Rate | ECG | Measures the total |
| | | number of heart beats |
| | | per minute |
| Heart Rhythm including P, | ECG | Measures the |
| Q, R, S, T analysis | | regularity of the |
| | | heartbeat. |
| Transthoracic impedance | ICG | Measures the electrical |
| | | impedance of the |
| | | thorax or chest of the |
| | | patient. |
| Skin Conductance | GSR | Measure the |
| | | conductance or |
| | | resistance of the skin |
| Blood oxygen levels | PPG | Volumetric changes in |
| | | arterial blood which is |
| | | associated with cardiac |
| | | activity, variations in |
| | | venous blood volume |
| Body or skin temperature | Temperature | Human body or skin |
| | | temperature |

### Step 3—Computing a Change Assessment Metric Clustering

Dimensionality reduction may be performed on this data using statistical techniques such as principal component analysis (PCA) and choosing the number of principal components needed to explain at least 95% of the variability within the data. Clustering methods can be applied to the data in the principal components' dimension to form clusters. The number of clusters may be determined empirically to find optimal separation of clusters. Clustering is then performed with the choice of optimal clusters to compute the cluster centroids.

After the formulation of clusters, there may be two approaches to determining an assessment metric. One leading directly to a quantitative assessment. Alternatively, there may be an intermediate qualitative assessment which could then be extended into a quantitative metric.

### Quantitative Metric—Compute Cluster Membership Distribution

Each aggregated minute of data is then sorted and clustered based on the computed centroids from the clustering step. An aggregate membership count for each cluster may be determined at different epochs of time from a few minutes to a day. The cluster counts can then be normalized to a fixed maxima of 100 so that the maximum cluster membership count is always 100.

A metric that tracks absolute change in cluster membership from one chosen epoch to the next is computed for each cluster using a normalized difference formula such as

$$Quantified {Change}_{i} = \vert \frac{{clustercount}_{i}^{n} - {clustercout}_{i}^{n - 1}}{{clustercount}_{i}^{n} + {clustercout}_{i}^{n - 1}} \vert$$

where, i is the cluster number, and n and n−1, refer to the member counts in the cluster for the current and previous epoch, respectively.

The mean Quantified Change across all the clusters may be used as a metric for absolute change trended from one chosen epoch to the next. This change may be used as an indicator of absolute change in patient status or evolution of the patient's state from the first day of recording to any subsequent day over the monitoring period.

### Qualitative Metric, or Qualitative Intermediate to a Quantitative Metric

The aggregated data for different epochs could be transformed into a graphical representation such as a scatter plot of the individual aggregated data points in the dimensions of the first 2 principal components or the two-dimensional self-organizing map (SOM) grid used to for the clusters. Alternatively, the scatter points may be presented as a contour or density map with colors representing the density of the aggregate points that occupy a particular region on the axes of the principal components or SOM grid. As such, these charts may be created to represent data spanning different durations of time such as a few seconds to years subject to the availability of data. Additionally, such charts could be created periodically spanning fixed durations of time epochs or specific time intervals to visualize the evolution of these charts from one epoch to the next. These charts could be presented as an animation with each frame representing a particular epoch. Similar to this exemplary visualization, additional multi-dimensional data visualization charts may be generated for qualitative assessment of patient status change.

Quantitative metrics could then be defined to represent the information present in these density of contour maps. For example—(a) geometric representations such as the dimensional attributes of a triangle such as base, height, and area, enclosing the histogram of cluster memberships or density associated with quadrants on the SOM grid, (b) minor and major axes of ellipses that are fit to the densest regions on the SOM grid.

Along with the measured data and the historic patient data, the systems and methods to manage and predict post-surgical recovery could use a plurality of patient reported data, including but not limited to, quality of life questions, SDOH (Social Determinants of Health) issues based questions, ADL (Activities of Daily Living) needs based, physical and spiritual well-being trends based questions. Such feedback from the patient may be obtained through a visual analog scale such as a scale from 0-100, or through multi-item questionnaires ranging from about 5 questions to 50 or more questions. Clinically validated objective measures can be derived from such questions or feedback soliciting methods. The questionnaires generally use a point scale as response. They may be multi-point scales like the Likert scale (for example:—positive items scale 1=‘none of the time’ to 5=‘all of the time’; for negative items the scale could be reversed) or Borg scale (ranging from 6-20; where 6 means no exertion, and 20 means maximal exertion). The questionnaires can cover various aspects of the patient's status such as emotional state, physical comfort, psychological support, physical independence, and pain. Such assessments are valuable in a complementary sense to the physical measures that are indicative of the patient's status in more directly quantifiable measures such as vital signs.

### CONCLUSION

In the preceding specification, the invention has been described with reference to specific exemplary embodiments and examples thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense.

Obvious variants of the disclosed embodiments are within the scope of the description and the claims that follow.

All references cited herein, as well as text appearing in the figures and tables, are hereby incorporated by reference in their entirety for all purposes to the same extent as if each were so individually denoted.

1-24. (canceled)

25. A method for assessment of a patient during perioperative care comprising the steps of:
a) selectively obtaining a plurality of input data from one or more measurement devices, selection and collection methods and/or processes;
b) subjecting the input data to a transformation selected from the group consisting of conditioning, feature engineering and combinations thereof;
c) translating the transformed input data into metrics; and
d) using the metrics obtained in step c to obtain an assessment of the patient.

26. The method of claim 25, wherein the input data is selected from the group consisting of past diagnoses, test results for blood biomarkers, proteins, metabolites, and/or cholesterol, biomedical vital signs collected from non-invasive medical devices, patient reported responses to Quality of Recovery questionnaires, physiological and biological data, a height of the patient, a weight of the patient, a gender of the patient, an age of the patient, a medical history and/or physical examination records of the patient, a medical status of the patient, a body mass index (BMI) of the patient, an ethnicity of the patient, a medical prescription history of the patient, a medical prescription status of the patient, types of treatments and medications received by the patient, types of medical treatments for health issues and insurance or claims information previously received by the patient, diet information for the patient, psychological history of the patient, a genetic indicator of the patient, biomarkers of the patient, the Electronic Medical Record of the patient information and combinations thereof.

27. The method of claim 26, wherein the physiological and biological data is selected from the group consisting of electrocardiogram, electromyogram, electrooculogram, electroencephalogram, galvanic skin resistance, goniometric measurements of all joint angles, absolute inclination, and orientation of different parts of the body in multiple axes, photoplethysmographic measures at various sites on the body, heart sounds, lung sounds, gastrointestinal sounds, joint sounds, acoustic impedance, electromagnetic impedance, ultrasonic impedance, blood oxygen levels, temperatures measured at different locations of the body, sweat biomarkers measured at various areas of the body such as lactate, pH, alcohol, nicotine, sodium, glucose, urea, chloride, discrete blood, interstitial fluid, cerebrospinal fluid (CSF) biomarker panels and combinations thereof.

28. The method of claim 25, wherein the input data comprises measurements made from patients reflective of physiological conditions selected from the group consisting of electrocardiogram, electromyogram, electroencephalogram, phonocardiogram, activity and posture, sweat, blood and urine analysis results, and historical information on diagnosed conditions, past surgical interventions, and history of medications.

29. The method of claim 26, wherein the biomedical vital signs are collected from non-invasive nanosensor medical devices.

30. The method of claim 26, wherein the data conditioning is obtained by methods selected from the group consisting of filtering, trend removal when there are gradual drifts in the measurement values due to the instrumentation used to perform the measurement, signal processing methods that increase the proportion of physiologically relevant data to the noise, transformations of the input data from the time domain to other domains, application of filtering techniques to segment and extract quantitative or qualitative measures correlated with physiological factors in turn correlated to patient status assessments, neural networks and combinations thereof.

31. The method of claim 25, wherein the input data is conditioned and after conditioning, the input data is prepared by translating the input data into a format that is compatible with Step c.

32. The method of claim 25, wherein the input data transformation is obtained by methods selected from the group consisting Fourier, wavelet, short-time Fourier, cepstral analysis, empirical mode decomposition, or wavelet decomposition.

33. The method of claim 25, wherein the feature engineering is comprised of feature extraction to result in features and wherein the feature extraction involves a technique or method selected from the group consisting of discrete Fourier and short-term fourier transforms, discrete cosine transform, autoregressive models, autoregressive moving average models, classes of linear predictive coding models, cepstral analysis derived mel-frequency cepstral coefficients, kernel-based modeling, multiresolution analysis using discrete and continuous wavelet transformations, wavelet packet transformations and decompositions, empirical mode decompositions, power spectrum estimation using techniques that measure spectral coupling across different signal modalities, non-negative matrix factorization, ambiguity kernel functions, a subset of the layers from a pre-trained multilayer neural networks used as a transformation from input data into feature vectors in the feature space, unsupervised or supervised clustering methods such as adaptive resonance-based neural networks, self-organizing maps, k-means clustering, k-nearest neighbors, Gaussian mixture models, and Naïve Bayes classifiers which group together similar feature sets by plurality of features extracted or plurality of statistically summarized inputs and assign group labels to each instance of a set of features.

34. The method of claim 33, wherein the feature extraction involves the use of multiresolution analysis and signal decomposition using wavelet transforms to condition heart sound data.

35. The method of claim 25, wherein the assessment provides an overall metric that is reflective of the patient's state of recovery, a risk stratification score or number that is reflective of a probability or likelihood of a patient developing symptoms of a complication or risk of developing a condition that requires emergency treatment following a surgical procedure or combinations thereof.

36. The method of claim 25, wherein the transformation in Step b is obtained by clustering methods and the translation of the transformed input data into metrics of Step c involves a mathematical model to transform a cluster membership into a one-dimensional metric.

37. The method of claim 25, further comprising a personalization method comprising:
a. performing one or more improvement, conditioning, and/or correction methods or processes to account for data quality and confounders;
b. performing data conditioning methods and processes for data conditioning and preparation of the data;
c. performing one or more feature extraction methods or processes to extract a plurality of features for signal and model assessment from one or more measurement devices and historic patient data;
d. performing normalization, combination, and/or transformation methods;
processes for the signal and model assessment to provide inputs for the assessment for improvements, conditioning, and correction; and
f. using the output of step d to provide a personalized assessment of a perioperative patient.

38. The method of claim 25, wherein the feature engineering comprises the steps of feature transformation and/or decomposition.

39. The method of claim 38, wherein the feature engineering further comprises feature selection.

40. The method of claim 38, wherein the transformation and/or decomposition involves techniques selected from the group consisting of box cox transformation, eigen value, vector decomposition, principal component analysis (PCA), kernel PCA, truncated singular value decomposition, multidimensional scaling, isometric mapping, t-distributed stochastic neighbor embedding, wavelet denoising, neural networks and combinations thereof.

41. The method of claim 39, wherein the method of feature selection is selected from the group consisting of measurement of mutual information using Kullback-Leibler convergence, minimum redundancy maximum relevance, impurity-based feature importance using random forest regression models, F-statistic or f-test, neighborhood component analysis, backward elimination, forward selection, permutation feature importance, factor analysis, and relief algorithm for regression.

42. The method of claim 25, wherein during Step b) the input data is conditioned using an engineering system selected from the group consisting of filtering in time, frequency, wavelet, or other domains defined by a span of output of a convolutional neural network prior to a final layer which is a connected layer, so that the transformation does not remove any information from the data that is being transformed, then transforming the conditioned input data into qualitative or quantitative metrics using a method selected from the group consisting of dimensionality reduction techniques consisting of box cox transformation, eigenvalue, and vector decomposition, principal component analysis (PCA), backward elimination, forward selection, random forests impurity-based importance, permutation feature importance, factor analysis, linear discriminant analysis, truncated singular value decomposition, kernel PCA, t-distributed stochastic neighbor embedding, multidimensional scaling, isometric mapping and combinations thereof and wherein the assessment of the patient using the metrics of Step d is a representation of the time varying status of a patient and indicates whether there has been a change in the overall status of the patient as a cumulative effect of changes that are manifesting among the metrics that were computed and chosen as relevant to tracking recovery after a surgery.

43. The method of claim 25, wherein based on the assessment, the method provides further actions selected from the group consisting of planning, support, follow-up, patient compliance, recovery prediction and tracking, potential treatment modifications and combinations thereof.

44. The method of claim 25, wherein the assessment is presented as a numeric, symbolic, image, or video.

45. The method of claim 25, wherein the method further comprises a continuous improvement method comprising:
a. performing improvements, conditioning, and/or correction methods and processes to the input data to account for data quality and confounders;
b. performing feature extraction methods and processes to the product of step b to extract a plurality of features for signal and model assessment from a plurality of measurement devices and historic patient data;
c. performing a plurality of feature selection methods and processes for selecting features that are relevant to the assessment;
d. performing one or more normalization, combination and/or transformation methods or processes for the signal and model assessment to provide inputs for the patient status assessment model for improvements, conditioning, and correction.

46. The method of claim 45, wherein the method further includes personalizing the assessment.

47. The method of claim 45, wherein the method further includes continuous improvement of the assessment through incorporation of patient specific data as part of the input data to improve the assessment.

48. The method of claim 45, wherein, a model is pre-trained on a population of at least 50 patients wherein the method is repeated and continuously improved upon by adding further input data obtained from the patient each time the method is repeated to generate a prediction model that is unique for the patient.

49. The method of claim 45, wherein, the method is repeated and wherein each time the method is repeated, updated input data obtained from the patient is added to step (a) resulting in an assessment model that is unique for the patient.

50. The method of claim 45, wherein the further input data is selected from the group consisting of patient reported outcomes, physiological measures, and psychological measures and combinations thereof.

51. The method of claim 45, wherein the method predicts a degree of certainty of from about 75% to about 95% for each patient status assessment associated therewith, wherein each of the degrees of confidence is based at least on predicted data, historical data, and/or patient questionnaire data.

52. The method of claim 45, wherein a generative neural network is added, wherein the generative neural network comprises a generator component and a discriminator component.

53. The method of claim 37, wherein the input data applied to the discriminator component generates a set of features that are used to train another neural network or machine learning model.

54. A method for improving a patient's recovery using assessment predictions generated during perioperative care comprising
a) selectively obtaining a plurality of input data from one or more measurement devices, selection and collection methods and/or processes;
b) subjecting the input data to a transformation selected from the group consisting of conditioning, feature engineering and combinations thereof;
c) translating the transformed input data into metrics; and
d) using the metrics obtained in Step c to obtain an assessment of the patient.
wherein the assessment is further configured to predict an outcome of a set of possible further surgeries for the patient at a specific point in time after the surgery; wherein the further configured assessment predictions for possible further surgeries are configured to predict a degree of confidence for each of the assessment predictions, where the degree of confidence indicates the likelihood that the patient will achieve the assessment prediction.

55. The method of claim 45, wherein the assessment predictions are based at least on measured data, derived data, extracted data, patient historic data, and/or patient questionnaire data.

56. The method of claim 45, wherein a report is generated assessing the status of the patient.

57. The method of claim 45, wherein the assessment prediction also provides treatment recommendations based on input data obtained from the patient post-surgery combined with the assessment predictions.

58. The method of claim 45, wherein the assessment prediction also provides intervention planning, support, follow-up, patient compliance, recovery prediction and tracking, and potential treatment modification options.

59. The method of claim 45, wherein input data and/or derivatives are obtained from a method selected from the group consisting of electrical activity based metrics, bioimpedance based metrics, goniometric measurements of all joint angles, absolute inclination, and orientation of different parts of the body in multiple axes, photoplethysmographic measures at various sites on the body, heart sounds, lung sounds, gastrointestinal sounds, and joint sounds, blood oxygen levels, skin and/or body temperatures measured at different locations of the body, biological parameters, geographic location and altitude metrics, patient historic data, patient questionnaires, risk stratification metrics by means of a hazard ratio or index, or a recovery percentage score indicative of change and trajectory of change in a patient's status around an index or event which involves a surgical intervention or combinations thereof, wherein the biological parameters are selected from the group consisting of lactate, pH, alcohol, sodium, glucose, urea, chloride, discrete blood, interstitial fluid, cerebrospinal fluid biomarker panels, metabolic panels and combinations thereof.
